(19) United States
(12) Patent Application Publication
(10) Pub. No.: US 2007/0231295 A1
(43) Pub. Date: Oct. 4, 2007

(54) Antimicrobial Silicon Oxide Flakes

(76) Inventors: Holger Hoppe, Lorrach (DE); Patrice Bujard, Courtepin (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/579,431

(22) PCT Filed: May 2, 2005
(86) PCT No.: PCT/EP05/51981

§ 371 Date: Nov. 2, 2006

(30) Foreign Application Priority Data
May 12, 2004 (EP) 04102069.4
Jul. 15, 2004 (EP) 04103383.8

(51) Int. Cl. A01N 59/00; A61K 31/02; A61K 31/74

(52) U.S. Cl. 424/78.09; 424/724; 514/770

(57) ABSTRACT

The present invention relates to SiO_{z} flakes, especially porous SiO_{z} flakes, wherein 0.70≦z≦2.0, especially 0.95≦z≦2.0, comprising an organic, or inorganic antimicrobial compound, or composition, which provide enhanced (long term) antimicrobial efficacy.

The present invention relates to SiO_{z} flakes, especially porous SiO_{z} flakes, wherein 0.70≦z≦2.0, especially 0.95≦z≦2.0, especially porous SiO₂ flakes, comprising an organic, or inorganic antimicrobial compound, or composition, which provide enhanced (long term) antimicrobial efficacy.

WO03/068868 describes the production of SiO₂ flakes having a thickness in the range from 20 to 2000 nm. Production involves the production of SiO_{y} flakes by PVD and oxidation of the SiO_{y} flakes by an oxygen-containing gas to SiO₂ flakes. The SiO₂ flakes can be provided with one or more metal oxide and/or metal layers, such as, for example, Cr, Ti, Mo, W, Al, Cu, Ag, Au, or Ni. In addition, pigments are described, which can be produced, for example by PVD of a three layer structure, SiO_{y}/substrate/SiO_{y} (0.95≦y≦1.8) and then heating of the three layer structure in a carbon containing gas, wherein the substrate is, for example, transition metals having a melting point greater than 1000° C., like Mo, Nb, Zr, Ti, Hf and W.

WO2004020530 relates to a cosmetic and personal care preparation or formulation comprising a gloss pigment comprising (a1) a core consisting of a substantially transparent or metallically reflecting material, and (a2) at least one coating substantially consisting of one or more silicon oxides, the molar ratio of oxygen to silicon being on average from 0.03 to 0.95. The metallic refelecting material is selected from Ag, Al, Au, Cu, Cr, Ge, Mo, Ni, Si, Ti, Zn, or alloys thereof.

WO2004/035684 describes plane parallel pigments having a SiOₓ core 0.03≦x≦0.95), a SiO_{z} layer (0.95≦y≦2.0) and a Layer D^{M} which includes metals or alloys thereof. The metals are selected from Ag, Al, Au, Cu, Co, Cr, Fe, Ge, Mo, Nb, Ni, Si, Ti, V, or alloys thereof,

WO03/106569 relates to plane-parallel pigments, comprising a silicon/silicon oxide layer obtainable by heating a SiO_{y} layer in an oxygen-free atmosphere at a temperature above 400° C., wherein 0.70≦y≦1.8, comprising

(a) a silicon/silicon oxide substrate layer obtainable by heating a SiO_{y} layer in an oxygen-free atmosphere at a temperature above 400° C., wherein 0.70≦y≦1.8,

a semi-transparent metal layer. Suitable metals for the semi-transparent metal layer are, for example, Cr, Ti, Mo, W, Al, Cu, Ag, Au, or Ni.

EP0960911 relates to pigment mixtures comprising (a) silicon dioxide (SiO₂) flakes coated with metal oxides and/or metals and (b) a colorant or filler in the form of platelet-shaped, needle-shaped or spherical particles. The metal is selected from Cr, Ti, Mo, W, Al, Cu, Ag, Au, or Ni.

WO2004/065295 (prior art pursuant to Art. 54 (3) and (4) EPC) describes a process for the production of porous SiO_{z} flakes (0.70≦z≦2.0). The SiO_{z} flakes appear to be ideal for supporting catalytic metals, such as copper or nickel based reforming catalysts, or palladium based catalysts for the Suzuki reaction. These particles have very high surface areas (˜700 m²/g), and nanoscale (2-50 nm) porosity.

JP3081209 discloses antimicrobial agents excellent in transparency of films even by blending thereof with a synthetic resin film without any bad influence on the transparency of the films, capable of being blended in various ingredients and exhibiting antimicrobial effects on diverse various germs such as Escherichia coil by supporting an antimicrobial metal on fine powdery silica. The antimicrobial agent is obtained by supporting 1-15 wt. %, preferably 2-10 wt. % antimicrobial metal, e.g. silver, copper, zinc, mercury, lead, bismuth, cadmium, chromium or thallium, in form of a salt on fine powdery silica. All examples relate to a process, which comprises immersing of a metal salt into silica and filtering. No addition of reducing agents is mentioned.

JP1268764 discloses a powder obtained by supporting an antimicrobial metal (e.g., metallic copper) on particle surfaces of an inorganic or extender pigment (e.g., zinc oxide or magnetite) consisting essentially of at least one element of Al, Ba, Ca, Cd, Co, Cr, Fe, Mg, Pb, Si, Sb or Zn.

A number of metal ions have been shown to possess antimicrobial activity, including silver, copper, zinc, mercury, tin, lead, bismuth, cadmium, chromium and thallium ions. It is theorized that these antimicrobial metal ions exert their effects by disrupting respiration and electron transport systems upon absorption into bacterial or fungal cells. Silver ions have been impregnated in the surfaces of medical implants, as described in U.S. Pat. No. 5,474,797. Silver ions have also been incorporated in catheters, as described in U.S. Pat. No. 5,520,664. The products described in these patents, however, do not exhibit an antimicrobial effect for a prolonged period of time because a passivation layer typically forms on the silver ion coating. This layer reduces the release rate of the silver ions from the product, resulting in lower antimicrobial effectiveness.

Antimicrobial zeolites can be prepared by replacing all or part of the ion-exchangeable ions in zeolite with antimicrobial metal ions, as described in U.S. Pat. Nos. 4,011,898; 4,938,955; 4,906,464; and 4,775,585. Polymers incorporating antimicrobial zeolites have been used to make refrigerators, dish washers, rice cookers, plastic films, chopping boards, vacuum bottles, plastic pails, and garbage containers. Other materials in which antimicrobial zeolites have been incorporated include flooring, wall paper, cloth, paint, napkins, plastic automobile parts, bicycles, pens, toys, sand, and concrete. Examples of such uses are described in U.S. Pat. Nos. 5,714,445, 5,697,203, 5,562,872, 5,180,585, 5,714,430, and 5,102,401.

U.S. Pat. No. 5,305,827 describes an antimicrobial hydrophilic coating for heat exchangers. The coating includes silver oxide, to inhibit microbial growth and improve adhesion to the heat transfer surfaces of a heat exchanger. However, this coating exhibits severe discoloration and is typically antimicrobially effective for 3 days or less.

Japanese Pat. Application No. 03347710 relates to a non-woven fabric bandage containing synthetic fibers and hydrophilic fibers. The synthetic fibers contain zeolite which is ion-exchanged with silver, copper, or zinc ions.

U.S. Pat. No. 4,923,450 discloses incorporating zeolite in bulk materials. When zeolite is conventionally compounded into polymers, however, the zeolite often aggregates, causing poor dispersion of the zeolite in the polymer. When such material is molded or extruded, the surface of the polymer is frequently beaded instead of flat. Poor dispersion of the zeolite also can cause changes in the bulk properties of the polymer, such as a reduction in tensile strength. U.S. Pat. No. 4,938,958 describes antimicrobial zeolites in which a portion of the ion-exchangeable ions in the zeolite are replaced with ammonium. This results in a product which exhibits reduced discoloration.

Inorganic particles, such as the oxides of titanium, aluminum, zinc and copper, may be coated with a composition which confers antimicrobial properties, for example, by releasing antimicrobial metal ions such as silver ions, which are described, e.g., in U.S. Pat. No. 6,444,726. Inorganic soluble glass particles containing antimicrobial metal ions, such as silver, are described, e.g., in U.S. Pat. Nos. 5,766,611 and 5,290,544.

Accordingly, it is the object of the present invention to provide antimicrobial particles having high antimicrobial activity.

Said object has been solved by SiO_{z} flakes, especially porous SiO_{z} flakes, wherein 0.70≦z≦2.0, especially 0.95≦z≦2.0, comprising an organic, or inorganic antimicrobial compound, or composition.

The antimicrobial SiO_{z} flakes of this invention are useful, because they are safe, if biocompatible antimicrobial metals are used, and have good affinity for a living body, in the fields of foods, living body materials, cosmetics, fibers, celluloses, coatings, plastics, filters, water absorption polymers etc., where antimicrobial properties are needed.

The term “SiO_{z} with 0.70≦z≦2.0” means that the molar ratio of oxygen to silicon at the average value of the silicon oxide substrate is from 0.70 to 2.0. The composition of the silicon oxide substrate can be determined by ESCA (electron spectroscopy for chemical analysis). The stoichiometry of silicon and oxygen of the silicon oxide substrate can be determined by RBS (Rutherford-Backscattering).

“Antimicrobial metals” are metals whose ions have an anti-microbial effect and which are preferably biocompatible. Preferred biocompatible anti-microbial metals include Ag, Au, Pt, Pd, Ir (i.e. the noble metals), Sn, Cu, Sb, Bi and Zn, with Ag being most preferred.

“Antimicrobial effect” means inhibition of bacterial (or other microbial) growth, or killing of microorganism.

The term “comprising silver” includes the combination of silver with other metals, such as, for example, zinc, copper and zirconium.

According to the present invention the term “SiO_{z} flakes comprising an antimicrobial compound, or composition” includes that the whole surface of the (porous) SiO_{z} flakes is covered by the antimicrobial compound, or composition, that the pores or parts of the pores of the porous SiO_{z} flakes are filled with the antimicrobial compound, or composition, and/or that the (porous) SiO_{z} flakes are coated at individual points with the antimicrobial compound, or composition. The latter two options are preferred against the first option. In one preferred embodiment, the pores or parts of the pores of the porous SiO_{z} flakes are filled with the antimicrobial compound, or composition. As the size of the pores of the SiO_{z} flakes can be controlled by the process for the production of the porous SiO_{z} flakes to be in the range of from ca. 1 to ca. 50 nm, especially ca. 2 to ca. 20 nm, it is, for example, possible to create nanosized metal particles within the pores of SiO_{z} flakes. In another preferred embodiment of the present invention, individual particles of the antimicrobial compounds, such as silver, having a particle size in the range of from 1 to 50 nm, especially 2 to 20 nm, are bonded to the surface of the antimicrobial (porous) SiO_{z} flakes. Hence, antimicrobial flakes are preferred comprising individual particles of the antimicrobial metals, such as silver, having a particle size in the range of from 1 to 50 nm, especially 2 to 20 nm.

The plate-like (plane-parallel) SiO_{z} structures (SiO_{z} flakes), especially porous SiO_{z} flakes used according to the present invention have a length of from 1 μm to 5 mm, a width of from 1 μm to 2 mm, and a thickness of from 20 nm to 1.5 μm, and a ratio of length to thickness of at least 2:1, the particles having two substantially parallel faces, the distance between which is the shortest axis of the particles. The porous SiO_{z} flakes are mesoporous materials, i.e. have pore widths of ca. 1 to ca. 50 nm, especially 2 to 20 nm. The pores are randomly inter-connected in a three-dimensional way. So, when used as a support, the passage blockage, which frequently occurs in SiO₂ flakes having a two-dimensional arrangement of pores can be prevented. The specific surface area of the porous SiO_{z} flakes depends on the porosity and ranges from ca. 400 m²/g to more than 1000 m²/g. Preferably, the porous SiO_{z} flakes have a specific surface area of greater than 500 m²/g, especially greater than 600 m²/g. The BET specific surface area is determined according to DIN 66131 or DIN 66132 (R. Haul und G. Dümbgen, Chem.-Ing.-Techn. 32 (1960) 349 and 35 (1063) 586) using the Brunauer-Emmet-Teller method (J. Am. Chem. Soc. 60 (1938) 309).

The SiO_{z} flakes, especially porous SiO_{z} flakes are not of a uniform shape. Nevertheless, for purposes of brevity, the flakes will be referred to as having a “diameter.” The SiO_{z} flakes have a plane-parallelism and a defined thickness in the range of ±10%, especially ±5% of the average thickness. The SiO_{z} flakes have a thickness of from 20 to 2000 nm, especially from 100 to 500 nm. It is presently preferred that the diameter of the flakes is in a preferred range of about 1-60 μm with a more preferred range of about 5-40 μm and a most preferred range of about 5-20 μm. Thus, the aspect ratio of the flakes of the present invention is in a preferred range of about 2.5 to 625 with a more preferred range of about 50 to 250.

The present invention is illustrated in more detail on the basis of the porous SiO_{z} flakes, but not limited thereto. Non-porous SiO_{z} flakes, which can be prepared according to a process described in WO04/035693, are also suitable.

The porous SiO_{z} flakes are obtainable by a process described in WO2004/065295. Said process comprises the steps of:

a) vapor-deposition of a separating agent onto a carrier to produce a separating agent layer,

b) the simultaneous vapor-deposition of SiO_{y} and a separating agent onto the separating agent layer (a),

c) the separation of SiO_{y} from the separating agent, wherein 0.70≦y≦1.80.

The platelike material can be produced in a variety of distinctable and reproducible variants by changing only two process parameters: the thickness of the mixed layer of SiO_{y} and separating agent and the amount of the SiO_{y} contained in the mixed layer.

The term “SiO_{y} with 0.70≦y≦1.80” means that the molar ratio of oxygen to silicon at the average value of the silicon oxide layer is from 0.70 to 1.80. The composition of the silicon oxide layer can be determined by ESCA (electron spectroscopy for chemical analysis). The stoichiometry of silicon and oxygen of the silicon oxide layer can be determined by RBS (Rutherford-Backscattering).

The separating agent vapor-deposited onto the carrier in step a) may be a lacquer (surface coating), a polymer, such as, for example, the (thermoplastic) polymers, in particular acryl- or styrene polymers or mixtures thereof, as described in U.S. Pat. No. 6,398,999, an organic substance soluble in organic solvents or water and vaporisable in vacuo, such as anthracene, anthraquinone, acetamidophenol, acetylsalicylic acid, camphoric anhydride, benzimidazole, benzene-1,2,4-tricarboxylic acid, biphenyl-2,2-dicarboxylic acid, bis(4-hydroxyphenyl)sulfone, dihydroxyanthraquinone, hydantoin, 3-hydroxybenzoic acid, 8-hydroxyquinoline-5-sulfonic acid monohydrate, 4-hydroxycoumarin, 7-hydroxycoumarin, 3-hydroxynaphthalene-2-carboxylic acid, isophthalic acid, 4,4-methylene-bis-3-hydroxynaphthalene-2-carboxylic acid, naphthalene-1,8-dicarboxylic anhydride, phthalimide and its potassium salt, phenolphthalein, phenothiazine, saccharin and its salts, tetraphenylmethane, triphenylene, triphenylmethanol or a mixture of at least two of those substances. The separating agent is alternatively an inorganic salt soluble in water and vaporisable in vacuo (see, for example, DE 198 44 357), such as sodium chloride, potassium chloride, lithium chloride, sodium fluoride, potassium fluoride, lithium fluoride, calcium fluoride, sodium aluminium fluoride and disodium tetraborate.

In detail, a salt, for example NaCl, followed successively by a layer of silicon suboxide (SiO_{y}) and separating agent, especially NaCl or an organic separating agent, is vapor-deposited onto a carrier, which may be a continuous metal belt, passing by way of the vaporisers under a vacuum of <0.5 Pa.

The mixed layer of silicon suboxide (SiO_{y}) and separating agent is vapor-deposited by two distinct vaporizers, which are each charged with one of the two materials and whose vapor beams overlap, wherein the separating agent is contained in the mixed layer in an amount of 1 to 60% by weight based on the total weight of the mixed layer.

The thicknesses of salt vapor-deposited are about 20 nm to 100 nm, especially 30 to 60 nm, those of the mixed layer from 20 to 2000 nm, especially 50 to 500 nm depending upon the intended characteristics of the product.

The carrier is immersed in a dissolution bath (water). With mechanical assistance, the separating agent layer rapidly dissolves and the product layer breaks up into flakes, which are then present in the solvent in the form of a suspension The porous silicon oxide flakes can advantageously be produced using an apparatus described in U.S. Pat. No. 6,270,840.

The suspension then present in both cases, comprising product structures and solvent, and the separating agent dissolved therein, is then separated in a further operation in accordance with a known technique. For that purpose, the product structures are first concentrated in the liquid and rinsed several times with fresh solvent in order to wash out the dissolved separating agent. The product, in the form of a solid that is still wet, is then separated off by filtration, sedimentation, centrifugation, decanting or evaporation.

A SiO_{1.00-1.8} layer is formed preferably from silicon monoxide vapour produced in the vaporiser by reaction of a mixture of Si and SiO₂ at temperatures of more than 1300° C.

A SiO_{0.70-0.99} layer is formed preferably by evaporating silicon monoxide containing silicon in an amount up to 20% by weight at temperatures of more than 1300° C.

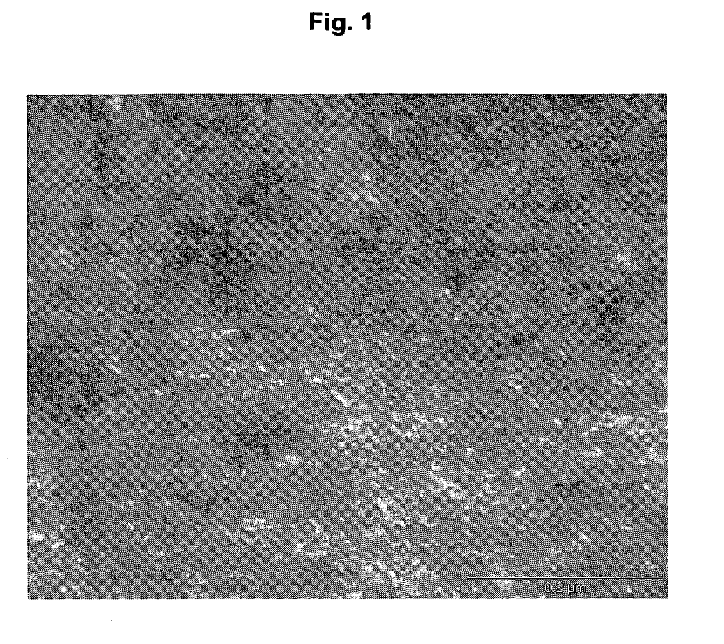

FIG. 1 is a TEM (transmission electron microscope) micrograph of the porous SiO_{z} (z=1.4-1.6) flakes used in Example 1 of the present application. The TEM micrograph shows that the pore size of the SiO_{z} flakes is in the range of about 4 nm.

The production of porous SiO_{z} flakes with z>1 can be achieved by providing additional oxygen during the evaporation. For this purpose the vacuum chamber can be provided with a gas inlet, by which the oxygen partial pressure in the vacuum chamber can be controlled to a constant value.

Alternatively, after drying, the product can be subjected to oxidative heat treatment. Known methods are available for that purpose. Air or some other oxygen-containing gas is passed through the plane-parallel structures of SiO_{y} wherein y is, depending on the vapor-deposition conditions, from 0.70, especially 1 to about 1.8, which are in the form of loose material or in a fluidised bed, at a temperature of more than 200° C., preferably more than 400° C. and especially from 500 to 1000° C. After several hours all the structures will have been oxidised to SiO_{z}. The product can then be brought to the desired particle size by means of grinding or air-sieving, wherein comminution of the fragments of film to pigment size can be effected, for example, by means of ultrasound or by mechanical means using high-speed stirrers in a liquid medium, or after drying the fragments in an air-jet mill having a rotary classifer.

Alternatively, after drying, the porous SiO_{y} particles can be heated according to WO03/106569 in an oxygen-free atmosphere, i.e. an argon or helium atmosphere, or in a vacuum of less than 13 Pa (10⁻¹ Torr), at a temperature above 400° C., especially 400 to 1100° C., whereby porous silicon oxide flakes containing Si nanoparticles can be obtained.

It is assumed that by heating SiO_{y} particles in an oxygen-free atmosphere, SiO_{y} disproportionates in SiO₂ and Si:
SiO_{y}→(y/y+a) SiO_{y+a}+(1−y/y+a) Si

In this disproportion porous SiO_{y+a} flakes are formed, containing (1−(y/y+a)) Si, wherein 0.70≦y≦1.8, especially 0.70≦y≦0.99 or 1≦y≦1.8, 0.05≦a≦1.30, and the sum y and a is equal or less than 2.SiO_{y+a} is an oxygen enriched silicon suboxide.
SiO_{y}→(y/2) SiO₂+(1−(y/2)) Si

The porous SiO_{z} flakes should have a minimum thickness of 50 nm, to be processible. The maximum thickness is dependent on the desired application, but is in general in the range of from 150 to 500 nm. The porosity of the flakes ranges from 5 to 85%.

The porous SiO_{z} flakes, comprise an organic, or inorganic antimicrobial compound, or composition.

In one embodiment of the present invention the antimicrobial compound, or composition is an organic antimicrobial compound, or composition. Examples of antimicrobial compounds are dimethyldimethylol hydantoin (Glydant®), methylchloroisothiazolinone/methylisothiazolinone (Kathon CG®), imidazolidinyl urea (Germall 115®, diazolidinyl urea (Germaill II®), benzyl alcohol, 2-bromo-2-nitropropane-1,3-diol (Bronopol®), formalin (formaldehyde), iodopropenyl butylcarbamate (Polyphase P100®), chloroacetamide, methanamine, methyldibromonitrile glutaronitrile (1,2-Dibromo-2,4-dicyanobutane or Tektamer®), glutaraldehyde, 5-bromo-5-nitro-1,3-dioxane (Bronidox®), phenethyl alcohol, o-phenylphenol/sodium o-phenylphenol, sodium hydroxymethylglycinate (Suttocide A®), polymethoxy bicyclic oxazolidine (Nuosept C®), dimethoxane, thimersal, dichlorobenzyl alcohol, captan, chlorphenenesin, dichlorophene, chlorbutanol, glyceryl laurate,halogenated diphenyl ethers,2,4,4′-trichloro-2′-hydroxy-diphenyl ether (Triclosan®, or TCS), 4,4′-dichloro-2′-hydroxydiphenyl ether, 2,2′-dihydroxy-5,5′-dibromo-diphenyl ether, phenolic compounds, phenol, 2-methyl phenol, 3-methyl phenol, 4-methyl phenol, 4-ethyl phenol, 2,4-dimethyl phenol, 2,5-dimethyl phenol, 3,4-dimethyl phenol,2,6-dimethyl phenol, 4-n-propyl phenol, 4-n-butyl phenol, 4-n-amyl phenol, 4-tert-amyl phenol, 4-n-hexyl phenol, 4-n-heptyl phenol, mono- and poly-alkyl and aromatic halophenols, p-chlorophenol, methyl p-chlorophenol,ethyl p-chlorophenol, n-propyl p-chlorophenol, n-butyl p-chlorophenol, n-amyl p-chlorophenol, sec-amyl p-chlorophenol, cyclohexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, o-benzyl p-chlorophenol, o-benxyl-m-methyl p-chlorophenol, o-benzyl-m, m-dimethyl p-chlorophenol, o-phenylethyl p-chlorophenol, o-phenylethyl-m-methyl p-chlorophenol, 3-methyl p-chlorophenol, 3,5-dimethyl p-chlorophenol, 6-ethyl-3-methyl p-chlorophenol, 6-n-propyl-3-methyl p-chlorophenol, 6-iso-propyl-3-methyl p-chlorophenol, 2-ethyl-3,5-dimethyl p-chlorophenol, 6-sec-butyl-3-methyl p-chlorophenol, 2-iso-propyl-3,5-dimethyl p-chlorophenol, 6-diethylmethyl-3-methyl p-chlorophenol, 6-iso-propyl-2-ethyl-3-methyl p-chlorophenol, 2-sec-amyl-3,5-dimethyl p-chlorophenol, 2-diethylmethyl-3,5-dimethyl p-chlorophenol, 6-sec-octyl-3-methyl p-chlorophenol, p-chloro-m-cresol,p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2-phenyl phenol.4-chloro-2-methyl phenol,4-chloro-3-methyl phenol, 4-chloro-3,5-dimethyl phenol, 2,4-dichloro-3,5-dimethylphenol, 3,4,5,6-terabromo-2-methylphenol, 5-methyl-2-pentylphenol, 4-isopropyl-3-methylphenol,para-chloro-meta-xylenol (pcmx), chlorothymol, phenoxyethanol, phenoxyisopropanol, 5-chloro-2-hydroxydiphenylmethane, resorcinol and its derivatives, resorcinol, methyl resorcinol, ethyl resorcinol, n-propyl resorcinol, n-butyl resorcinol, n-amyl resorcinol, n-hexyl resorcinol, n-heptyl resorcinol, n-octyl resorcinol, n-nonyl resorcinol, phenyl resorcinol, benzyl resorcinol, phenylethyl resorcinol, phenylpropyl resorcinol, p-chlorobenzyl resorcinol, 5-chloro 2,4-dihydroxydiphenyl methane, 4′-chloro 2,4-dihydroxydiphenyl methane, 5-bromo 2,4-dihydroxydiphenyl methane, 4′-bromo 2,4-dihydroxydiphenyl methane, bisphenolic compounds, 2,2′-methylene bis(4-chlorophenol), 2,2′-methylene bis(3,4,6-trichlorophenol), 2,2′-methylene bis-(4-chloro-6-bromophenol), bis(2-hydroxy-3,5-dichlorophenyl)sulphide, bis(2-hydroxy-5-chloro-benzyl)sulphide, benzoic esters (parabens), methylparaben, propylparaben, butylparaben, ethylparaben, isopropylparaben, isobutylparaben, benzylparaben, sodium methylparaben, sodium propylparaben, halogenated carbanilides, 3,4,4′-trichlorocarbanilides (Triclocarban® or TCC), 3-trifluoromethyl-4,4′-dichlorocarbanilide, 3,3′,4-trichlorocarbanilide, chlorohexidine and its digluconate, diacetate and dihydrochloride, undecenoic acid, hexetidine, and poly(hexamethylenebiguanide) hydrochloride (Cosmocil®). Antifungal agents are, for example, selected from the group consisting of thiabendazole, 10,10′ oxybisphenoxyarsine, tebuconazole, tolnaftate, zinc bis-(2-pyridinethiol-1-oxide), 2n-octyl-4-isothiazolin-3-one, 4,5-dichloro-octyl-4-isothiazoline, N-butyl-benzisothiazoline, 3-iodo-2-propinylbutylcarbamate, methyl-1H-benzimidazol-2-ylcarbamate and mixtures thereof.

Incorporation of the antimicrobial compound, or composition into the pores of the SiO_{z} flakes can be achieved by diffusion, precipitation, covalent bonding and/or ion exchange.

The SiO_{z} flakes comprising an organic antimicrobial compound can be obtained by a method, which comprises

a) dispersing the SiO_{z} flakes in a solution of the organic antimicrobial compound, adding the SiO_{z} flakes to a solution of the organic antimicrobial compound or adding the organic antimicrobial compound to a dispersion of the SiO_{z} flakes,

b) optionally precipitating the organic antimicrobial compound onto the SiO_{z} flakes, and

c) isolating the SiO_{z} flakes comprising the organic antimicrobial compound.

Preference is given to a method, which comprises

a) adding the SiO_{z} flakes to a solution of the organic antimicrobial compound,

b) optionally precipitating the organic antimicrobial compound onto the SiO_{z} flakes, and

c) subsequently isolating the SiO_{z} flakes comprising the organic antimicrobial compound.

Advantageously, the procedure is such that the organic antimicrobial compound is first dissolved in a suitable solvent (I) and then the SiO_{z} flakes are dispersed in the resulting solution. It is, however, also possible, vice versa, for the SiO_{z} flakes first to be dispersed in the solvent (I) and then for the organic antimicrobial compound to be added and dissolved.

Any solvent that is miscible with the first solvent and that so reduces the solubility of the organic antimicrobial compound, that it is completely, or almost completely, deposited onto the substrate is suitable as solvent (II). In this instance, both inorganic solvents and also organic solvents come into consideration. Isolation of the coated substrate can then be carried out in conventional manner by filtering off, washing and drying.

In a further embodiment of the invention, the antimicrobial compound, or composition comprises an antimicrobial metal salt. Said metal salt comprises metals selected from the group consisting of Groups I (A, B), II (A, B), III A, IV(A,B), VIB, VIII, rare earth compounds, and combinations thereof. More preferably, metal salts include salts of metals selected from the group consisting of Mn, Ag, Au, Zn, Sn, Fe, Cu, Al, Ni, Co, Ti, Zr, Cr, La, Bi, K, Cd, Yb, Dy, Nd, Ce, Tl, Pr, and combinations thereof. Even more preferably, metal salts include salts of metals selected from the group consisting of Mn, Ag, Au, Zn, Sn, Fe, Cu, Al, Ni, Co, Ti, Zr, Cr, La, and combinations thereof. Most preferably, the metal salts include salts of metals selected from the group consisting of Ag, Au, Cu, Zn, and combinations thereof.

More particularly, the metal salts include, but are not limited to, metal chelates and salts like bishistidine complexes, bromides, chondroitin sulfate, chromites, cyanides, dipiocolinates, ethylhexanoates, glycerolate complex, methoxides, polyphosphonates, paraphenolsulfonates, perchlorates, phenolsulfonates, selenides, stearates, thiocyanates, tripolyphosphates, tungstates, phosphates, carbonates, para-amihobenzoate, paradimethylaminobenzoates, hydroxides, para-methoxycinnamate, naphthenates, stearates, caprates, laurates, myristates, palmitates, oleates, picolinates, pyrithiones, fluorides, aspartates, gluconates, iodides, oxides, nitrites, nitrates, phosphates, pyrophosphates, sulfides, mercaptopyridine-oxides (e.g., zinc pyrithione), nicotinates, and nicotinamides, hinokitiol, acetates, ascorbates, chlorides, benzoates, citrates, fumarates, gluconates, glutarates, lactates, malates, malonates, salicylates, succinates, sulfates, undecylates, and combinations thereof.

More preferably, the metal salts are selected from the group consisting of phosphates, carbonates, para-aminobenzoate, paradimethylaminobenzoates, hydroxides, para-methoxycinnamate, naphthenates, stearates, caprates, laurates, myristates, palmitates, oleates, picolinates, pyrithiones, fluorides, aspartates, gluconates, iodides, oxides, nitrites, nitrates, phosphates, pyrophosphates, sulfides, mercaptopyridine-oxides (e.g., zinc pyrithione), nicotinates, and nicotinamides, hinokitiol, acetates, ascorbates, chlorides, benzoates, citrates, fumarates, gluconates, glutarates, lactates, malates, malonates, salicylates, succinates, sulfates, undecylates and combinations thereof.

Even more preferably, the metal salts are selected from the group consisting of fluorides, aspartates, gluconates, iodides, oxides, nitrites, nitrates, phosphates, pyrophosphates, sulfides, mercaptopyridine- oxides (e.g., zinc pyrithione), nicotinates, and nicotinamides, hinokitiol, acetates, ascorbates, chlorides, benzoates, citrates, fumarates, gluconates, glutarates, lactates, malates, malonates, salicylates, succinates, sulfates, undecylates, and combinations thereof.

Even more preferably, the metal salts and complexes are: acetates, ascorbates, chlorides, benzoates, citrates, fumarates, gluconates, glutarates, lactates, malates, malonates, salicylates, succinates, sulfates, undecylates, and combinations thereof.

In a preferred embodiment the present invention is directed to SiO_{z} flakes comprising metal salts of benzoic acid analogs.

Preferred benzoic acid analogs include those having the structure 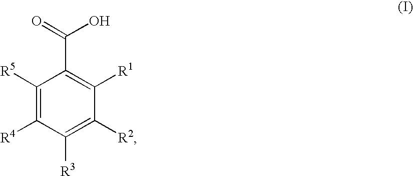
wherein R¹, R², R⁴, and R⁵ are independently selected from the group consisting of H, OH, F, I, Br, Cl, SH, NH₂, CN, alkyl, alkoxy, NR₂, OR, NO₂, COR, CONR₂, CO₂R, SO₃R′; R³ is independently selected from the group consisting H, OH, F, I, Br, Cl, SH, CN, alkyl, alkoxyl, OR, NO₂, COR, CONR₂, CO₂R, SO₃R; wherein R is independently selected from the group consisting H, alkyl, and aralkyl groups and R′ is R, or NR₂.

Suitable alkyl groups include saturated or unsaturated, linear or branched chain, substituted or unsubstituted alkyl groups, preferably C₁-C₄-, more preferably C₁-C₃-, most preferably C₁-C₂alkyl groups (preferably CH₃ or C₂H₅). Nonlimiting examples of substituted alkyls are CH₂CO₂R, CH₂OR, CH₂OR, CH₂COR, and CH₂NR₂, where R is defined as above.

Suitable aralkyl groups include substituted or unsubstituted aralkyl groups, preferably benzyl, which can be substituted by one or more C₁-C₄alkyl, or C₁-C₄alkoxy groups.

Suitable alkoxy groups include saturated or unsaturated, linear or branched chain, substituted or unsubstituted alkoxy groups, preferably C₁-C₄-, more preferably C₁-C₃-, most preferably C₁-C₂alkoxyl groups (preferably CH₃ or C₂H₅).

Preferred halogens are selected from the group consisting of I, Br and Cl.

Preferred benzoic acid analogs are those wherein R¹, R², R⁴, and R⁵ are independently selected from the group consisting of H, hydroxy, amino, diethylamino, dimethylamino, methyl, ethyl, propyl, butyl, ethoxy, methoxy, propoxy, butoxy, C(O)CH₃, C(O)C₃H₇, C(O)C₄H₈, CO₂CH₃, CO₂C₃H₇, CH₂OCH₃, CH₂OC₃H₇, COOH, chloro, fluoro, bromo, trifluoromethyl, nitro, and cyano. R³ is selected from the group consisting H, hydroxy, diethylamino, dimethylamino, methyl, ethyl, propyl, butyl, ethoxy, methoxy, propoxy, butoxy, C(O)CH₃, C(O)C₃H₇, C(O)C₄H₈, CO₂CH₃, CO₂C₃H₇, CH₂OH, CH₂OCH₃, CH₂OC₃H₇, COOH, chloro, fluoro, bromo, trifluoromethyl, nitro, and cyano.

Examples of these benzoic acid analogs are selected from the group consisting of benzoic acid, salicylic acid, 2-nitrobenzoic acid, thiosalicylic acid, 2,6-dihydroxybenzoic acid, 3-hydroxybenzoic acid, 5-nitrosalicylic acid, 5-bromosalicylic acid, 5-iodosalicylic acid, 5-fluorosalicylic acid, 3-chlorosalicylic acid, 4-chlorosalicylic acid, 5-chlorosalicylic acid, phthalic acid, and combinations thereof.

Most preferably, the benzoic acid analog is selected from the group consisting of salicylic acid, benzoic acid, and combinations thereof.

The selection of the metal ion and the corresponding anion is dependent on the particular use. Antimicrobial metal ions of silver, gold, copper and zinc, in particular, are considered safe even for in vivo use. Antimicrobial silver ions are particularly useful for in vivo use due to the fact that they are not substantially absorbed into the body. Such salts include silver acetate, silver benzoate, silver carbonate, silver ionate, silver iodide, silver lactate, silver laureate, silver oxide, silver palmitate, silver protein, and silver sulfadiazine.

In a further preferred embodiment, the present invention is directed to SiO_{z} flakes comprising tetrasilver tetroxide, i.e., silver (I, III) oxide, and derivatives thereof, especially tetrasilver tetroxide (Ag₄O₄).

The tetrasilver tetroxide containing porous SiO_{z} flakes can be obtained by a process comprising:

a) providing an aqueous solution containing a water soluble silver salt, such as silver nitrate;

b) contacting said SiO_{z} flakes with said solution for a period of time sufficient to uniformly wet said SiO_{z} flakes with said solution;

c) immersing said wetted SiO_{z} flakes in a bath containing a second aqueous solution containing a strong alkali, such as sodium hydroxide, and a water soluble oxidizing agent, such as sodium persulfate, and heating said bath for a period of time sufficient to precipitate tetrasilver tetroxide on said SiO_{z} flakes; and

d) removing said SiO_{z} flakes from said bath.

Isolation of the coated substrate can then be carried out in conventional manner by filtering off, washing and drying.

In a particularly preferred embodiment of the present invention the antimicrobial compound, or composition comprise a metal, especially a metal which is selected from Mn, Ag, Zn, Sn, Fe, Cu, Al, Ni, Co, Ti, Zr, Cr, La, Bi, K, Cd, Yb, Dy, Nd, Ce, Tl, Pr and combinations thereof, very especially silver, gold, copper, zinc, and combinations thereof.

The metal containing SiO_{z} flakes can be obtained by

a) suspending the SiO_{z} flakes in a solvent,

b) adding solvent soluble antimicrobial metal salts and optionally a reducing agent to the solution,

c) isolation of the metal containing SiO_{z} flakes.

Alternatively, SiO_{z} flakes can be added to a solution of the metal salt and a reducing agent can optionally be added to the solution. Isolation of the coated substrate can then be carried out in conventional manner by filtering off, washing and drying.

Alternatively, the metal comprising SiO_{z} flakes are obtainable by a process described in WO2004/065295. Said process comprises the steps of:

a) vapor-deposition of a separating agent onto a carrier to produce a separating agent layer,

b) the simultaneous vapor-deposition of SiO_{y} and a metal, especially silver, onto the separating agent layer (a),

c) the separation of SiO_{y} from the separating agent, wherein 0.70≦y≦1.80. However, said process is less preferred and the antimicrobial SiO_{z} flakes of the present invention are preferably obtained by a wet chemical coating process.

The methods for preparing the antimicrobial metal containing SiO_{z} flakes will hereunder be explained in more detail, especially on the basis of silver as metal:

The SiO_{z} flakes are suspended in an aqueous and/or organic solvent containing medium in the presence of a metal compound and metal compound is deposited onto the substrate by addition of a reducing agent, if the SiO_{z} flakes themselves do not act as reducing agent, or porous SiO₂ flakes are used as substrate. The metal compound is, for example, silver nitrate, copper chloride, palladium chloride, nickel acetate, or nickel acetyl acetonate. Nickel chloride can be used as metal compound and hypophosphite can be used as reducing agent. In case of silver nitrate the following compounds can be used as reducing agents: aldehydes (formaldehyde, acetaldehyde, benzalaldehyde), ketones (acetone), carbonic acids and salts thereof (tartaric acid, ascorbinic acid), reductones (isoascorbinic acid, triosereductone, reductine acid), and reducing sugars (glucose).

In a preferred embodiment of the present invention the metal compound is, for example, copper chloride, palladium chloride, or nickel acetate. In said embodiment the metal compound is suspended in water/or organic solvent, especially water, and a solution of the metal salt is added under stirring. Then the suspension is optionally heated up to the boiling point of the solvent for 1 h to 2 days. The reducing agent, preferably hydrazine, or NaBH₄, is added to the cooled suspension. the suspension is optionally heated up to the boiling point of the solvent for 1 h to 2 days. The obtained SiO_{z} flakes are washed with water and/or another solvent, like a C₁-C₄alcohol, especially methanol or ethanol, sufficiently followed by drying. The SiO_{z} flakes are preferably dried at a temperature of 105° C. to 115° C. under normal pressure or at a temperature of 10° C. to 90° C. under reduced pressure (1 to 30 torr). The obtained SiO_{z} flakes can subsequently be calcined at 200 to 800° C, especially 200 to 600° C., wherein colourless metall coated SiO_{z} flakes can be obtained. While the present invention does not comprise porous SiO₂ flakes (BET 750 m²/g) obtained by heating the SiO₂ flakes and [(C₆H₄CH₂NMe₂-2)Pd(OAc)(PPh₃)] in 5 ml xylene under rigorous stirring and heating to reflux for 2 hours, it comprises their use as antimicobia.

In a particularly preferred embodiment of the present invention, the SiO_{z} flakes comprise silicon and/or silicon suboxide, i.e. silicon-silicon bonds are present, and work themselves as reducing agent, i.e. z<2, especially 1≦z≦1.8, very especially 1.4≦z≦1.8. In said embodiment the antimicrobial SiO_{z} flakes of the present invention may be obtained by bringing porous SiO_{z} flakes into contact with a previously prepared aqueous and/or organic solvent, especially an alcoholic solution containing anions and antimicrobial metal ions, such as silver, copper and zinc ions to cause deposition of the metal into the pores of the porous SiO_{z} flakes or onto the surface of the porous SiO_{z} flakes, or non-porous SiO_{z} flakes. The contact between these ions may be carried out according to a batch technique or a continuous technique (such as a column method) at a temperature of from −114° C. ° C. to 70° C., preferably from to −70° C. to 30° C., for 1 h to 8 days, especially 1 h to 2 days, very especially 1 to 12 hours optionally under an atmosphere of inert gas, such as nitrogen, or argon. For instance, there may be mentioned such a silver ion source as silver nitrate, silver sulfate, silver perchlorate, silver acetate, and diamine silver nitrate; such a copper ion source as copper(II) nitrate, copper sulfate, copper perchlorate, copper acetate, tetracyan copper potassium; and such a zinc ion source as zinc(II) nitrate, zinc sulfate, zinc perchlorate, zinc acetate and zinc thiocyanate. In case of silver nitrate the silver ions are reduced to silver by the silicon and/or silicon suboxide present in the (porous) SiO_{z} flakes, whereby silver nanoparticles, having a particle size in the range of 1 to 50 nm, especially 1 to 20 nm, very especially 2 to 10 nm, are formed in the pores or on the surface of the SiO_{z} flakes. Said silver nanoparticles have extremely antiseptic efficacy; wide antibacterial spectrum; high bactericidal effect, especially during contact with water; no toxicity and no irrigation. If the above reaction is carried out below 30 ° C., especially below −20° C., very especially at −40 to −60° C., and the obtained SiO_{z} flakes are subsequently calcined at 200 to 600° C., colourless silver coated SiO_{z} flakes can be obtained, which are especially suitable for use in applications, where transparent silver coated SiO_{z} flakes are required, such as, for example, contact lenses. In said aspect of the present invention the pore size of the porous SiO_{z} flakes, or in other words the particle size of the silver nanoparticles is in the range of 1 to 20 nm, especially 2 to 10 nm.

The wording silver nanoparticles having a particle size in the range of 1 nm to 50 nm means that, in general, at least 80 percent, especially 95 percent of the silver nanoparticles have a particle size in the range from 1 nm to 50 nm, wherein at least 50 percent of the silver nanoparticles have preferably a particle size in the range from 1 nm to 20 nm. Most preferably, at least 50 percent of the particles have a particle size in the range from 2 to 10 nm. The largest dimension (e.g. length) of the silver nanoparticles is measured to determine the particle size. Particle size is determined by an electron micrograph or by laser diffraction using a Fraunhofer diffraction instrument.

The content of the metal, such as silver, in the SiO_{z} flakes is generally 0.001 to 20.0 percent by weight, especially 0.01 to 10 percent by weight, very especially 0.1 to 5.0 percent by weight.

The content of the metal, such as silver, in the SiO_{z} flakes may properly be controlled by adjusting the concentration of each ion species (or salt) in the aforesaid aqueous mixed solution. For example, if the antimicrobial SiO_{z} flakes of the invention comprise nitrate and silver ions, the antimicrobial SiO_{z} flakes having a silver ion content of 0.1 to 5% can properly be obtained by bringing the SiO_{z} flakes into contact with an aqueous silver nitrate solution, or a solution of silver nitrate in a C₁-C₄alcohol, especially methanol or ethanol, having a silver ion concentration of 0.0001 M/I to 0.5 M/I, especially 0.01 M/I to 0.1 M/I.

Alternatively, the antimicrobial SiO_{z} flakes comprising different antimicrobial metals may be prepared by using separate aqueous and/or alcoholic solutions each containing single different metal ion species (or salt) and bringing the SiO_{z} flakes into contact with each solution one by one.

The SiO_{z} flakes thus treated are washed with water and/or another solvent, like a C₁-C₄alcohol, especially methanol or ethanol, sufficiently followed by drying. The SiO_{z} flakes are preferably dried at a temperature of 105° C. to 115° C. under normal pressure or at a temperature of 10° C. to 90° C. under reduced pressure (1 to 30 torr). Optionally the SiO_{z} flakes can subsequently be calcined at 200 to 600° C. In case of porous SiO_{z} flakes, comprising silver nanoparticles, calcining may cause a reduction of the particle size of the silver nanoparticles, whereas in case of the non-porous SiO_{z} flakes, comprising silver nanoparticles, calcining may lead to an agglomeration of the silver nanoparticles. It is assumed that during calcining silver present on the surface of the porous SiO_{z} flakes migrates into the pores of the porous SiO_{z} flakes by capillary action.

Antimicrobial non-porous SiO_{z} flakes can be obtained in an analogous manner. The term “SiO_{z} flakes” normally comprises “non-porous SiO_{z} flakes” as well as “porous SiO_{z} flakes”. However, porous SiO_{z} flakes are more preferred than non-porous SiO_{z} flakes.

The antimicrobial SiO_{z} flakes according to the present invention may be used in any fields in which the development and proliferation of microorganisms such as general bacteria, eumycetes and algae must be suppressed.

Hence, a further aspect of the present invention is directed to antimicrobial products, or compositions, comprising the aforementioned antimicrobial SiO_{z} flakes.

For example, in the field of water systems, the antimicrobial SiO_{z} flakes of the present invention may be used as antimicrobial and anti-algal agent in water cleaner, water of a cooling tower, and a variety of cooling water.

Likewise of particular interest is the use of the antimicrobial SiO_{z} flakes for thermoplastic or thermosetting coatings.

Substrates to be coated include wood, ceramic materials, metals, plastics, or articles coated or stained with organic materials.

The binder can in principle be any binder which is customary in industry, for example those described in Ullmann's Encyclopedia of Industrial Chemistry, 5th Edition, Vol. A18, pp. 368-426, VCH, Weinheim 1991. In general, it is a film-forming binder based on a thermoplastic or thermosetting resin, predominantly on a thermosetting resin. Examples thereof are alkyd, acrylic, polyester, phenolic, melamine, epoxy and polyurethane resins and mixtures thereof.

The binder can be a cold-curable or hot-curable binder; the addition of a curing catalyst may be advantageous. Suitable catalysts which accelerate curing of the binder are described, for example, in Ullmann's Encyclopedia of Industrial Chemistry, Vol. A18, p.469, VCH Verlags-gesellschaft, Weinheim 1991.

Preference is given to coating compositions in which the binder comprises a functional acrylate resin and a crosslinking agent.

Examples of coating compositions containing specific binders are:

1. paints based on cold- or hot-crosslinkable alkyd, acrylate, polyester, epoxy or melamine resins or mixtures of such resins, if desired with addition of a curing catalyst;

2. two-component polyurethane paints based on hydroxyl-containing acrylate, polyester or polyether resins and aliphatic or aromatic isocyanates, isocyanurates or polyisocyanates;

3. two-component polyurethane paints based on thiol-containing acrylate, polyester or polyether resins and aliphatic or aromatic isocyanates, isocyanurates or polyisocyanates;

4. one-component polyurethane paints based on blocked isocyanates, isocyanurates or polyisocyanates which are deblocked during baking, if desired with addition of a melamine resin;

5. one-component polyurethane paints based on aliphatic or aromatic urethanes or polyurethanes and hydroxyl-containing acrylate, polyester or polyether resins;

6. one-component polyurethane paints based on aliphatic or aromatic urethaneacrylates or polyurethaneacrylates having free amino groups within the urethane structure and melamine resins or polyether resins, if necessary with curing catalyst;

7. two-component paints based on (poly)ketimines and aliphatic or aromatic isocyanates, isocyanurates or polyisocyanates;

8. two-component paints based on (poly)ketimines and an unsaturated acrylate resin or a polyacetoacetate resin or a methacrylamidoglycolate methyl ester;

9. two-component paints based on carboxyl- or amino-containing polyacrylates and polyepoxides;

10. two-component paints based on acrylate resins containing anhydride groups and on a polyhydroxy or polyamino component;

11. two-component paints based on acrylate-containing anhydrides and polyepoxides;

12. two-component paints based on (poly)oxazolines and acrylate resins containing anhydride groups, or unsaturated acrylate resins, or aliphatic or aromatic isocyanates, isocyanurates or polyisocyanates;

13. two-component paints based on unsaturated polyacrylates and polymalonates;

14. thermoplastic polyacrylate paints based on thermoplastic acrylate resins or externally crosslinking acrylate resins in combination with etherified melamine resins;

15. paint systems based on siloxane-modified or fluorine-modified acrylate resins;

16. paint systems, especially for clearcoats, based on malonate- blocked isocyanates with melamine resins (e.g. hexamethoxymethylmelamine) as crosslinker (acid catalyzed);

17. UV-curable systems based on oligomeric urethane acrylates and/or acrylatacrylaten, if desired in combination with other oligomers or monomers;

18. dual cure systems, which are cured first by heat and subsequently by UV or electron irradiation, or vice versa, and whose components contain ethylenic double bonds capable to react on irradiation with UV light in presence of a photoinitiator or with an electron beam.

Coating systems based on siloxanes are also possible, e.g. systems described in WO 98/56852, WO 98/56853, DE-A-2914427, or DE-A4338361.

The coating composition can also comprise further components, examples being solvents, pigments, dyes, plasticizers, stabilizers, rheologic or thixotropic agents, drying catalysts and/or levelling agents. Examples of possible components are described in Ullmann's Encyclopedia of Industrial Chemistry, 5th Edition, Vol. A18, pp. 429-471, VCH, Weinheim 1991.

Possible drying catalysts or curing catalysts are, for example, free (organic) acids or bases, or (organic) blocked acids or bases which may be deblocked by thermal treatment or irradiation, organometallic compounds, amines, amino-containing resins and/or phosphines. Examples of organometallic compounds are metal carboxylates, especially those of the metals Pb, Mn, Co, Zn, Zr or Cu, or metal chelates, especially those of the metals Al, Ti, Zr or Hf, or organometallic compounds such as organotin compounds.

Examples of metal carboxylates are the stearates of Pb, Mn or Zn, the octoates of Co, Zn or Cu, the naphthenates of Mn and Co or the corresponding linoleates, resinates or tallates.

Examples of metal chelates are the aluminium, titanium or zirconium chelates of acetylacetone, ethyl acetylacetate, salicylaldehyde, salicylaldoxime, o-hydroxyacetophenone or ethyl trifluoroacetylacetate, and the alkoxides of these metals.

Examples of organotin compounds are dibutyltin oxide, dibutyltin dilaurate or dibutyltin dioctoate.

Examples of amines are, in particular, tertiary amines, for example tributylamine, triethanolamine, N-methyldiethanolamine, N-dimethylethanolamine, N-ethylmorpholine, N-methylmorpholine or diazabicyclooctane (triethylenediamine), diazabicycloundecene, DBN (=1,5-diazabicyclo[4.3.0]non-5-ene), and salts thereof. Further examples are quaternary ammonium salts, for example trimethylbenzylammonium chloride.

Amino-containing resins are simultaneously binder and curing catalyst. Examples thereof are amino-containing acrylate copolymers.

The curing catalyst used can also be a phosphine, for example triphenylphosphine.

The coating compositions can also be a radiation-curable coating compositions. In this case, the binder essentially comprises monomeric or oligomeric compounds containing ethylenically unsaturated bonds (prepolymers), which after application are cured by actinic radiation, i.e. converted into a crosslinked, high molecular weight form. Where the system is UV-curing, it generally contains at least one photoinitiator as well. Corresponding systems are described in the abovementioned publication Ullmann's Encyclopedia of Industrial Chemistry, 5th Edition, Vol. A18, pages 451-453.

The coating compositions can be applied to any desired substrates, for example to metal, wood, plastic or ceramic materials.

The coating compositions can be applied to the substrates by the customary methods, for example by brushing, spraying, pouring, dipping or electrophoresis; see also Ullmann's Encyclopedia of Industrial Chemistry, 5th Edition, Vol. A18, pp. 491-500.

Depending on the binder system, the coatings can be cured at room temperature or by heating. The coatings are preferably cured at 50-150° C., and in the case of powder coatings or coil coatings even at higher temperatures.

The coating compositions can comprise an organic solvent or solvent mixture in which the binder is soluble. The coating composition can otherwise be an aqueous solution or dispersion. The vehicle can also be a mixture of organic solvent and water. The coating composition may be a high-solids paint or can be solvent-free (e.g. a powder coating material). Powder coatings are, for example, those described in Ullmann's Encyclopedia of Industrial Chemistry, 5th Ed., A18, pages 438444. The powder coating material may also have the form of a powder-slurry (dispersion of the powder preferably in water).

The pigments can be inorganic, organic or metallic pigments.

The coating compositions may also contain further additives, such as for example light stabilizers as mentioned above. In particular UV-absorbers and sterically hindered amines are advantageously added.

In the field of paints, the antimicrobial SiO_{z} flakes of the present invention can impart antimicrobial, antifungus and anti-algal properties to coated films by directly mixing the antimicrobial SiO_{z} flakes with various kinds of paints such as lyophilic paints, lacquer, varnish, and alkyl resin type, aminoalkyl resin type, vinyl resin type, acrylic resin type, epoxy resin type, urethane resin type, water type, powder type, chlorinated rubber type, phenolic paints; or by coating the antimicrobial SiO_{z} on the surface of the coated films. In the field of construction, the antimicrobial SiO_{z} flakes of the invention may impart antimicrobial, antifungus and anti-algal properties to various parts for construction such as materials for joint and materials for wall and tile by admixing the antimicrobial SiO_{z} flakes with materials for parts for construction or applying the antimicrobial SiO_{z} flakes to the surface of such a material for construction. Applicable systems include decorative coatings (water- and solvent borne coatings), industrial coatings (coil coating and UV-curable coatings) and powder coatings and paints, especially PVC flooring, parquet flooring, gel-coats, adhesives and the like.

Hence, the present invention is also directed to high molecular weight organic materials, comprising the antimicrobial SiO_{z} flakes of the present invention.

Examples of the high molecular weight organic material include a thermoplastic or thermosetting resin such as polyethylene (for example LDPE, HDPE or MDPE), polypropylene, polyvinyl chloride (PVC), acrylonitrile-butadiene-styrene copolymer (ABS), nylons, polyesters, unsaturated polyesters (UP), polyvinylidene chloride, polyamides, styrene-acrylonitrile copolymers (SAN), polystyrene (PS), polymethyl methacrylate (PMMA), polyacrylnitrile (PAN), polyethylene terephthalate (PET), polyacetals, polyvinyl alcohol, polycarbonate, acrylic resins, fluoroplastics, polyurethane (PUR), thermoplastic polyurethane (TPU), phenolic resins, urea resins, meramine resins, unsaturated polyester resins, epoxy resins, urethane resins, rayon, urea formaldehyde resin (UF), cuprammonium rayon, acetates, triacetates, vinylidene, natural or synthetic rubbers.

Accordingly, the instant invention pertains also to an antimicrobial polymer composition comprising

A) a plastic resin, and

B) an effective antimicrobial amount of a mixture of the antimicrobial SiO_{z} flakes as described above.

In said embodiment the antibacterial metals for use in metal-containing SiO_{z} flakes preferably include silver, copper, zinc, mercury, tin, lead, bismuth, cadmium, chromium, cobalt, nickel, zirconium, or a combination of two or more of these metals. Preference is given to silver, copper, zinc and zirconium, or a combination of these. Especially preferred metals are silver alone or a combination of silver with copper, zinc or zirconium.

Preferably, the plastic resin is selected from the group consisting of polyethylene (for example LDPE, HDPE or MDPE), polypropylene, acrylonitrile-butadiene-styrene copolymer (ABS), styrene-acrylonitrile copolymer (SAN), polystyrene (PS), polymethyl methacrylate (PMMA), polyacryl nitrile (PAN), polyethylene terephthalate (PET), polycarbonate (PC), polyamide (e.g. PA6, PA6,6, PA6,12), polyvinyl chloride (PVC), polymer latex, polyurethane (PUR), thermoplastic polyurethane (TPU), urea formaldehyde resin (UF) and unsaturated polyester (UP).

The effective antimicrobial amount of component (B) is for example 0.005 to 10%, based on the weight of component (A).

The instant invention also pertains to plastic films, fibers or articles that comprise the novel antimicrobial SiO_{z} flakes (B).

The antimicrobial SiO_{z} flakes and optional further additives may be added to the plastic resin, e.g. the polyolefin, individually or mixed with one another. If desired, the individual components of an additive mixture can be mixed with one another in the melt (melt blending) before incorporation into the plastic material.

The incorporation of the antimicrobial SiO_{z} flakes and optional further additives into the plastic material is carried out by known methods such as dry mixing in the form of a powder, or wet mixing in the form of solutions or suspensions. The antimicrobial SiO_{z} flakes and optional further additives may be incorporated, for example, before or after molding or also by applying the dissolved or dispersed stabilizer mixture to the plastic material, with or without subsequent evaporation of the solvent. The antimicrobial SiO_{z} flakes and optional further additives can also be added to the plastic material in the form of a masterbatch which contains these components in a concentration of, for example, about 2.5% to about 70% by weight; in such operations, the polymer can be used in the form of powder, granules, solutions, suspensions or in the form of latices.

If added to a plastic resin in the form of a masterbatch or concentrate, the novel antimicrobial SiO_{z} flakes are added via carriers such as LDPE, HDPE, MDPE, PP, ABS, SAN, PS, acrylates, PMMA, polyamide, polyesters, PVC, latex, styrene, polyol, TPU, unsaturated esters, urea, paraformaldehyde, water emulsion, etc. The total concentration of a) +b) in the carriers is from about 2.5% to about 70% by weight based on the weight of the carrier.

The antimicrobial SiO_{z} flakes and optional further additives can also be added before, during or after polymerization or crosslinking.

The antimicrobial SiO_{z} flakes and optional further additives can be incorporated into the plastic material in pure form or encapsulated in waxes, oils or polymers.

The instant invention relates also to a process for stabilizing an antimicrobial polymer against discoloration which comprises incorporating into said polymer an effective antimicrobial amount of the antimicrobial SiO_{z} flakes as described above.

The plastic films, fibers and articles of the present invention are advantageously employed for applications that require long-term hygienic activity on the surface, e.g., medical devices, hand rails, door handles, mobile phones, keyboards etc. The antimicrobial plastic films, fibers and articles of the present invention are used for example in hospitals, households, public institutions, ventilation systems, air cleaning and air conditioning systems and waste disposal systems. Plastic articles exposed to outdoor weathering that may have incorporated therein antimicrobial SiO_{z} flakes of the present invention are for example waste containers, swimming pool equipment, outdoor swing set equipment, slides, playground equipment, water tanks, out door furniture, and the like, and stadium seats.

The plastic films, fibers and articles of the present invention exhibit high antimicrobial activity at the surface.

The compositions, plastic films, fibers and articles of the present invention, that is to say, the polymer substrates, may also have incorporated therein one or more known additives. Preferred additional additives are selected from the group consisting of antioxidants, ultra-violet light absorbers, hindered amines, phosphites or phosphonites, hydroxylamines, nitrones, benzofuran-2-ones, thiosynergists, polyamide stabilizers, metal stearates, nucleating agents, fillers, reinforcing agents, lubricants, emulsifiers, dyes, pigments, optical brighteners, flame retardants, antistatic agents and blowing agents.

The composition is prepared by incorporating the antimicrobial SiO_{z} flakes into the resin by means of kneading it with the antimicrobial SiO_{z} flakes or coating the antimicrobial SiO_{z} flakes on the surface of such a resin in order to impart antimicrobial, antifungus and anti-algal properties to each of these plastics. In order to provide antibacterial, antifungus and antialgal properties to the composition, the content of the antimicrobial SiO_{z} flakes suitably ranges from 0.05 to 80 wt %, preferably 0.1 to 10 wt %.

Polymers incorporating the antimicrobial SiO_{z} flakes can be used to make refrigerators, dish washers, rice cookers, plastic film, chopping boards, vacuum bottles, plastic pails, heat exchangers, bath tubs, table tops, conveyor belts and garbage containers. Other materials in which the antimicrobial SiO_{z} flakes can be incorporated include flooring, wall paper, cloth, paint, napkins, plastic automobile parts, bicycles, pens, toys, sand, and concrete. Examples of such uses are described in U.S. Pat. Nos. 5,714,445; 5,697,203; 5,562,872; 5,180,585; 5,714,430; U.S. Pat. No. 5,305,827 and 5,102,401.

In the field of paper making, the antimicrobial SiO_{z} flakes of the invention may be incorporated into various paper materials such as wet tissue paper, paper packaging materials, paper and paper board for packaging applications, wall paper, corrugated boards, a sheet of paper, paper for maintaining freshness by papermaking from a material therefor together with the antimicrobial SiO_{z} flakes; or by coating the resultant paper with the antimicrobial SiO_{z} flakes for the purpose of imparting antimicrobial and antifungus properties to these paper.

Additional carriers suitable for the antimicrobial SiO_{z} flakes of the present invention may include various substrate-based products. In such instances, the antimicrobial SiO_{z} flakes may be impregnated into or onto the substrate products. For instance, suitable carriers include, but are not limited to, dry and wet wipes suitable for personal care and household use (e.g., nonwoven baby wipes, household cleaning wipes, surgical preparation wipes, etc.); diapers; infant changing pads; dental floss; personal care and household care sponges or woven cloths (e.g., washcloths, towels, etc.); tissue-type products (e.g. facial tissue, paper towels, etc.); and disposable garments (e.g., gloves, smocks, surgical masks, infant bibs, socks, shoe inserts, etc.).

Furthermore, the antimicrobial SiO_{z} flakes of the present invention may be utilized in various product forms for personal care use including, but not limited to, chewing gum, toothpaste, mouthwash, skin care products like deodorants, lotions and creams, rinse-off products like soaps and shower gels etc. Similarly, the antimicrobial SiO_{z} flakes of the present invention may be incorporated into various household care products including, but not limited to, hard surface cleaners (e.g., disinfectant sprays, liquids, or powders); dish or laundry detergents (liquid or solid), floor waxes, glass cleaners, etc. Similarly, the antimicrobial SiO_{z} flakes of the present invention may be incorporated into cosmetic compositions, including but not limited to lotions, cleansers, creams, aqueous solutions, alcohol gels, tissues, wipes, etc.

The antimicrobial SiO_{z} flakes of the present invention are highly efficacious for household cleaning applications (e.g., hard surfaces like floors, countertops, tubs, dishes and softer cloth materials like clothing, sponges, paper towels, etc.), personal care applications (e.g. deodorants, lotions and creams, shower gels, soaps, shampoos, wipes) and industrial and hospital applications (e.g., sterilization of instruments, medical devices, gloves). These compositions are efficacious for rapidly cleaning surfaces which are infected or contaminated with microorganisms.

Accordingly, the present invention relates also to personal care products, such as hand soaps, hand sanitizers, body washes, shower gels, body lotions, and combinations thereof, or a household care product, such as hard surface cleaners, dish detergents, and floor waxes.

The antimicrobial SiO_{z} flakes according to the present invention are particularly suitable as antimicrobials in cosmetic personal care applications such as deodorants, skin, hair and oral care products and rinse off products.

Other important applications for the antimicrobial SiO_{z} flakes according to the present invention are home care applications for cleaning and disinfection of hard surfaces and fabric care applications such as liquid detergents and softeners.

Cosmetic or pharmaceutical preparations contain from 0.05-40% by weight, based on the total weight of the composition, of the antimicrobial SiO_{z} flakes of the present invention, especially antimicrobial SiO_{z} flakes comprising antimicrobial metal salts, or metals, especially silver, gold, copper, zinc and combinations thereof.

The antimicrobial SiO_{z} flakes of the present invention, especially the antimicrobial SiO_{z} flakes comprising antimicrobial metal salts described above, might posses antiviral efficacy. As used herein, “antiviral efficacy” refers to something capable of killing viruses such as influenza and Severe A cute Respiratory Syndrome (SARS). SARS is a respiratory tract viral infection that is believed to be the result of viral infection caused by a family of viruses known as coronaviruses, viruses typically associated with the common cold.

The cosmetic formulations or pharmaceutical compositions according to the present invention may additionally contain one or more than one further antimicrobial agent as listed below.

Examples of antimicrobials which can additionally be used in the present invention are: Pyrithiones, especially the zinc complex (ZPT), Octopirox®, Dimethyldimethylol Hydantoin (Glydant®), Methylchloroisothiazolinone/methylisothiazolinone (Kathon CG®),Sodium Sulfite, Sodium Bisulfite, Imidazolidinyl Urea (Germall 115® Diazolidinyl Urea (Germaill II®), Benzyl Alcohol, 2-Bromo-2-nitropropane-1,3-diol (Bronopol®), Formalin (formaldehyde), Iodopropenyl Butylcarbamate (Polyphase P100®), Chloroacetamide, Methanamine, Methyldibromonitrile Glutaronitrile (1,2-Dibromo-2,4-dicyanobutane or Tektamer®), Glutaraldehyde, 5-bromo-5-nitro-1,3-dioxane (Bronidox®), Phenethyl Alcohol, o-Phenylphenol/sodium o-phenyl-phenol, Sodium Hydroxymethylglycinate (Suttocide A®), Polymethoxy Bicyclic Oxazolidine (Nuosept C®), Dimethoxane, Thimersal, Dichlorobenzyl Alcohol, Captan, Chlorphenenesin, Dichlorophene, Chlorbutanol, Glyceryl Laurate,Halogenated Diphenyl Ethers,2,4,4′-trichloro-2′-hydroxy-diphenyl ether (Triclosan®. or TCS),2,2′-dihydroxy-5,5′-dibromo-diphenyl ether, Phenolic Compounds, Phenol, 2-Methyl Phenol, 3-Methyl Phenol, 4-Methyl Phenol, 4-Ethyl Phenol, 2,4-Dimethyl Phenol, 2,5-Dimethyl Phenol, 3,4-Dimethyl Phenol,2,6-Dimethyl Phenol, 4-n-Propyl Phenol, 4-n-Butyl Phenol, 4-n-Amyl Phenol, 4-tert-Amyl Phenol, 4-n-Hexyl Phenol, 4-n-Heptyl Phenol, Mono- and Poly-Alkyl and Aromatic Halophenols, p-Chlorophenol, Methyl p-Chlorophenol,Ethyl-p-Chlorophenol, n-Propyl p-Chlorophenol, n-Butyl p-Chlorophenol, n-Amyl p-Chlorophenol, sec-Amyl p-Chlorophenol, Cydohexyl p-Chlorophenol, n-Heptyl p-Chlorophenol, n-Octyl p-Chlorophenol, o-Chlorophenol, Methyl o-Chlorophenol, Ethyl o-Chlorophenol, n-Propyl o-Chlorophenol, n-Butyl o-Chlorophenol, n-Amyl o-Chlorophenol, tert-Amyl o-Chlorophenol, n-Hexyl o-Chlorophenol, n-Heptyl o-Chlorophenol, o-Benzyl p-Chlorophenol, o-Benxyl-m-methyl p-Chlorophenol, o-Benzyl-m, m-dimethyl p-Chlorophenol, o-Phenylethyl p-Chlorophenol, o-Phenylethyl-m-methyl p-Chlorophenol, 3-Methyl p-Chlorophenol, 3,5-Dimethyl p-Chlorophenol, 6-Ethyl-3-methyl p-Chlorophenol, 6-n-Propyl-3-methyl p-Chlorophenol, 6-iso-Propyl-3-methyl p-Chlorophenol, 2-Ethyl-3,5-dimethyl p-Chlorophenol, 6-sec-Butyl-3-methyl p-Chlorophenol, 2-iso-Propyl-3,5-dimethyl p-Chlorophenol, 6-Diethylmethyl-3-methyl p-Chlorophenol, 6-iso-Propyl-2-ethyl-3-methyl p-Chlorophenol, 2-sec-Amyl-3,5-dimethyl p-Chlorophenol, 2-Diethylmethyl-3,5-dimethyl p-Chlorophenol, 6-sec-Octyl-3-methyl p-Chlorophenol, p-Chloro-m-cresol,p-Bromophenol, Methyl p-Bromophenol, Ethyl p-Bromophenol, n-Propyl p-Bromophenol, n-Butyl p-Bromophenol, n-Amyl p-Bromophenol, sec-Amyl p-Bromophenol, n-Hexyl p-Bromophenol, Cyclohexyl p-Bromophenol, o-Bromophenol, tert-Amyl o-Bromophenol, n-Hexyl o-Bromophenol, n-Propyl-m,m-Dimethyl o-Bromophenol, 2-Phenyl Phenol.4-Chloro-2-methyl phenol,4-Chloro-3-methyl phenol, 4-Chloro-3,5-dimethyl phenol, 2,4-Dichloro-3,5-dimethylphenol, 3,4,5,6-Terabromo-2-methyl-phenol, 5-Methyl-2-pentylphenol, 4-Isopropyl-3-methylphenol,Para-chloro-meta-xylenol (PCMX), Chlorothymol, Phenoxyethanol, Phenoxyisopropanol, 5-Chloro-2-hydroxydiphenyl-methane, Resorcinol and its Derivatives, Resorcinol, Methyl Resorcinol, Ethyl Resorcinol, n-Propyl Resorcinol, n-Butyl Resorcinol, n-Amyl Resorcinol, n-Hexyl Resorcinol, n-Heptyl Resorcinol, n-Octyl Resorcinol, n-Nonyl Resorcinol, Phenyl Resorcinol, Benzyl Resorcinol, Phenylethyl Resorcinol, Phenylpropyl Resorcinol, p-Chlorobenzyl Resorcinol, 5-Chloro 2,4-Dihydroxydiphenyl Methane, 4′-Chloro 2,4-Dihydroxydiphenyl Methane, 5-Bromo 2,4-Dihydroxydiphenyl Methane, 4′-Bromo 2,4-Dihydroxydiphenyl Methane, Bisphenolic Compounds, 2,2′-Methylene bis(4-chlorophenol), 2,2′-Methylene bis(3,4,6-trichlorophenol), 2,2′-Methylene bis-(4-chloro-6-bromophenol), bis(2-hydroxy-3,5-dichlorophenyl)sulphide, bis(2-hydroxy-5-chlorobenzyl)sulphide, Benzoic Esters (Parabens), Methylparaben, Propylparaben, Butylparaben, Ethylparaben, Isopropylparaben, Isobutylparaben, Benzylparaben, Sodium Methylparaben, Sodium Propylparaben, Halogenated Carbanilides, 3,4,4′-Trichlorocarbanilides (Triclocarban® or TCC), 3-Trifluoromethyl4,4′-dichlorocarbanilide, and 3,3′,4-Trichlorocarbanilide.

Another class of antibacterial agents, which can additionally be used, are the so-called “natural” antibacterial actives, referred to as natural essential oils. These actives derive their names from their natural occurrence in plants. Typical natural essential oil antibacterial actives include oils of anise, lemon, orange, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, gum benzoin, basil, fennel, fir, balsam, menthol, ocmea origanum, Hydastis carradensis, Berberidaceae daceae, Ratanhiae and Curcuma longa. Also included in this class of natural essential oils are the key chemical components of the plant oils which have been found to provide the antimicrobial benefit. These chemicals include, but are not limited to anethol, catechole, camphene, carvacol, eugenol, eucalyptol, ferulic acid, farnesol, hinokitiol, tropolone, limonene, menthol, methyl salicylate, thymol, terpineol, verbenone, berberine, ratanhiae extract, caryophellene oxide, citronellic acid, curcumin, nerolidol and geraniol.

Additional active agents are antibacterial metal salts. This class generally includes salts of metals in groups 3b-7b, 8 and 3a-5a. Specifically are the salts of aluminum, zirconium, zinc, silver, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thulium, ytterbium, lutetium and mixtures thereof.

Combinations with chelating agents can also improve the antimicrobial activity of the antimicrobial agents of the present invention. Examples for such chelating agents resulting in additional antimicrobial effects or synergistic activity when combined with the antimicrobal agent of formula (I) are ethylene di-amine tetra acetic acid (EDTA), beta-alanine diacetic acid (EDETA), hydroxyethylene di-amino tetraacetic acid, nitrilotriacetic acid (NTA) and ethylenediamine disuccinic acid (S,S-EDDS, R,R-EDDS or S,R-EDDS).

The antimicrobial compositions of the present invention comprise from about 0.05% to about 10%, preferably from about 0.1% to about 2%, and more preferably from about 0.2% to about 1%, by weight of the deansing composition, of an anionic surfactant.

Non-limiting examples of anionic lathering surfactants useful in the compositions of the present invention are disclosed in McCutcheon's, Detergents and Emulsifiers, North American edition (1990), published by The Manufacturing Confectioner Publishing Co.; McCutcheon's, Functional Materials, North American Edition (1992); and U.S. Pat. No. 3,929,678, to Laughlin et al., issued Dec. 30, 1975, all of which are incorporated by reference.

A wide variety of anionic surfactants are potentially useful herein. Non-limiting examples of anionic lathering surfactants include those selected from the group consisting of alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, and mixtures thereof. Mixtures of anionic surfactants can be used effectively in the present invention.

The antimicrobial composition of the present invention may further comprise a non-ionic surfactant. Typical nonionic surfactants are condensated products of ethylene oxide with various reactive hydrogen-containing compounds reactive therewith having long hydrophobic chains (e.g. aliphatic chains of about 12-20 carbon atoms), which condensation products (“ethoxamers”) contain hydrophilic polyoxyethylene moieties, such as condensation products of poly(ethyleneoxide) with fatty acids, fatty alcohols, fatty amides, polyhydric alcohols (e.g. sorbitan monostearate) and polypropylene oxide (e.g. Pluronic ® materials). Polyoxamers are e.g. block copolymers of polyoxyethylene and polyoxypropylene having an average molecular weight from about 3000 to 5000 and a preferred average molecular weight from about 3500 to 4000 and containing about 10-80% hydrophilic polyoxyethylene groups, by weight, of the block copolymer (e.g. Pluronic F127).

The antimicrobial composition of the present invention may further comprise an amphoteric surfactant. As amphoteric surfactants C₈-C₁₈-betains, C₈-C₁₈-sulfobetains, C₈-C₂₄-alkylamido-C₁-C₄-alkylene betains, imidazoline carboxylates, alkylamphocarboxycarbonic acids, alkylamphocarbonic acid (e.g. lauroamphoglycinate) and N-alkyl-β-aminopropionate or -iminodipropionate can be used, in particular the C₁₀-C₂₀-alkylamidoC₁-C₄-alkylenbetaine and coco fatty acid amide propylbetaine.

The antimicrobial composition of the present invention may also comprise a proton donating agent, preferably from about 0.1% to about 10%, more preferably from about 0.5% to about 8%, and most preferably from about 1% to about 5%, based on the weight of the composition, of a proton donating agent. By “proton donating agent” it is meant any acid compound or mixture thereof, which results in undissociated acid on the skin after use. Proton donating agents can be organic acids, including polymeric acids, mineral acids or mixtures thereof.

The pH of the antimicrobial compositions of the present invention must be adjusted to a sufficiently low level in order to either form or deposit substantial undissociated acid on the skin. The pH of the present composition should be adjusted and preferably buffered to a range from about 3.0 to about 6.0, preferably from about 3.0 to about 5.0 and more preferably from about 3.5 to about 4.5.

In order to achieve the mildness required of the antimicrobial composition of the present invention, optional ingredients to enhance the mildness to the skin can be added. These ingredients include cationic and nonionic polymers, co-surfactants, moisturizers and mixtures thereof. Polymers useful herein include polyethylene glycols, polypropylene glycols, hydrolyzed silk proteins, hydrolyzed milk proteins, hydrolyzed keratin proteins, guar hydroxypropyltrimonium chloride, polyquats, silicone polymers and mixtures thereof. When used, the mildness enhancing polymers comprise from about 0.1% to about 1%, preferably from about 0.2% to about 1.0%, and more preferably from about 0.2% to about 0.6%, by weight of the antimicrobial composition.

Another group of mildness enhancers are lipid skin moisturizing agents which provide a moisturizing benefit to the user when the lipophilic skin moisturizing agent is deposited to the user's skin. When used in the antimicrobial personal cleansing compositions herein, lipophilic skin moisturizing agents are used, they are employed at a level of about 0.1% to about 30%, preferably from about 0.2% to about 10%, most preferably from about 0.5% to about 5% by weight of the composition.

A wide variety of lipid type materials and mixtures of materials are suitable for use in the antimicrobial compositions of the present invention. Preferably, the lipophilic skin conditioning agent is selected from the group consisting of hydrocarbon oils and waxes, silicones, fatty acid derivatives, cholesterol, cholesterol derivatives, di- and tri-glycerides, vegetable oils, vegetable oil derivatives, liquid nondigestible oils such as those described in U.S. Pat. No. 3,600,186 to Mattson; Issued Aug. 17, 1971 and U.S. Pat. Nos. 4,005,195 and 4,005,196 to Jandacek et al; both issued Jan. 25, 1977, all of which are herein incorporated by reference, or blends of liquid digestible or nondigestible oils with solid polyol polyesters such as those described in U.S. Pat. No. 4,797,300 to Jandacek; issued Jan.10, 1989; U.S. Pat. Nos. 5,306,514, 5,306,516 and 5,306,515 to Letton; all issued Apr. 26, 1994, all of which are herein incorporated by reference, and acetoglyceride esters, alkyl esters, alkenyl esters, lanolin and its derivatives, milk tri-glycerides, wax esters, beeswax derivatives, sterols, phospholipids and mixtures thereof. Fatty acids, fatty acid soaps and water soluble polyols are specifically excluded from our definition of a lipophilic skin moisturizing agent.

The antimicrobial compositions of the present invention can comprise a wide range of optional ingredients. The CTFA International Cosmetic Ingredient Dictionary, Sixth Edition, 1995, which is incorporated by reference herein in its entirety, describes a wide variety of nonlimiting cosmetic and pharmaceutical ingredients commonly used in the skin care industry, which are suitable for use in the compositions of the present invention. Nonlimiting examples of functional classes of ingredients are described at page 537 of this reference.

Examples of these functional classes include: abrasives, anti-acne agents, anticaking agents, antioxidants, binders, biological additives, bulking agents, chelating agents, chemical additives, colorants, cosmetic astringents, cosmetic biocides, denaturants, drug astringents, emulsifiers, external analgesics, film formers, fragrance components, humectants, opacifying agents, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, UV absorbers, and viscosity increasing agents (aqueous and nonaqueous). Examples of other functional classes of materials useful herein that are well known to one of ordinary skill in the art include solubilizing agents, sequestrants, and keratolytics, and the like.

Examples for antioxidants are amino acids or amino acid derivatives, imidazoles and their derivatives, peptides such as D,L-carnosin, carotinoids, carotines and their derivatives, liponic acid, metal chelating agents (such as alpha-hydroxy fatty acids, palmitinic acid, phytinic acid, lactoferrine), alpha-hydroxyacids (e.g. citric acid, lactic acid, maleic add), humic acid, gallate, EDTA, EGTA and their derivatives, unsaturated fatty acids and their derivatives, vitamine C and its derivatives, rutinic acid and its derivatives, alpha-glycosyl rutin, ferulic acid, butylhydroxytoluol, butyihydroxyanisol and suitable derivatives of these substances.

UV absorbers in the formulations might be those listed in the Table below:

Suitable UV filter substances which can be used in the
antimicrobal compositions of the present invention

p-aminobenzoic acid derivatives, for example 4-dimethylaminobenzoic acid 2-ethylhexyl ester;
salicylic acid derivatives, for example salicylic acid 2-ethylhexyl ester;
benzophenone derivatives, for example 2-hydroxy-4-methoxybenzophenone and its 5-sulfonic
acid derivative;
dibenzoylmethane derivatives, for example 1-(4-tert-butylphenyl)-3-(4-methoxyphenyl)-
propane-1,3-dione;
diphenylacrylates, for example 2-ethylhexyl 2-cyano-3,3-diphenylacrylate, and 3-(benzofura-
nyl) 2-cyanoacrylate;
3-imidazol-4-ylacrylic acid and esters;
benzofuran derivatives, especially 2-(p-aminophenyl)benzofuran derivatives, described in
EP-A-582 189, US-A-5 338 539, US-A-5 518 713 and EP-A-613 893;
polymeric UV absorbers, for example the benzylidene malonate derivatives described in
EP-A-709 080;
cinnamic acid derivatives, for example the 4-methoxycinnamic acid 2-ethylhexyl ester and
isoamyl ester or cinnamic acid derivatives described in US-A-5 601 811 and WO 97/00851;
camphor derivatives, for example 3-(4′-methyl)benzylidene-bornan-2-one, 3-benzylidene-
bornan-2-one, N-[2(and 4)-2-oxyborn-3-ylidene-methyl)-benzyl]acrylamide polymer, 3-(4′-
trimethylammonium)-benzylidene-bornan-2-one methyl sulfate, 3,3′-(1,4-
phenylenedimethine)-bis(7,7-dimethyl-2-oxo-bicyclo[2.2.1]heptane-1-methanesulfonic acid)
and salts, 3-(4′-sulfo)benzylidene-bornan-2-one and salts; camphorbenzalkonium
methosulfate;
hydroxyphenyltriazine compounds, for example 2-(4′-methoxyphenyl)-4,6-bis(2′-hydroxy-4′-
n-octyloxyphenyl)-1,3,5-triazine; 2,4-bis{[4-(3-(2-propyloxy)-2-hydroxy-propyloxy)-2-
hydroxy]-phenyl}-6-(4-methoxyphenyl)-1,3,5-triazine; 2,4-bis{[4-(2-ethyl-hexyloxy)-2-
hydroxy]-phenyl}-6-[4-(2-methoxyethyl-carboxyl)-phenylamino]-1,3,5-triazine; 2,4-bis{[4-
(tris(trimethylsilyloxy-silylpropyloxy)-2-hydroxy]-phenyl}-6-(4-methoxyphenyl)-1,3,5-triazine;
2,4-bis{[4-(2″-methylpropenyloxy)-2-hydroxy]-phenyl}-6-(4-methoxyphenyl)-1,3,5-triazine;
2,4-bis{[4-(1′,1′,1′,3′,5′,5′,5′-heptamethyltrisilyl-2″-methyl-propyloxy)-2-hydroxy]-phenyl)-6-(4-
methoxyphenyl)-1,3,5-triazine; 2,4-bis{[4-(3-(2-propyloxy)-2-hydroxy-propyloxy)-2-hydroxy]-
phenyl}-6-[4-ethylcarboxy)-phenylamino]-1,3,5-triazine;
benzotriazole compounds, for example 2,2′-methylene-bis(6-(2H-benzotriazol-2-yl)-4-
(1,1,3,3-tetramethylbutyl)-phenol;
trianilino-s-triazine derivatives, for example 2,4,6-trianiline-(p-carbo-2′-ethyl-1′-oxy)-1,3,5-
triazine and the UV absorbers disclosed in US-A-5 332 568, EP-A-517 104, EP-A-507 691,
WO 93/17002 and EP-A-570 838;
2-phenylbenzimidazole-5-sulfonic acid and salts thereof;
menthyl o-aminobenzoates;
physical sunscreens coated or not as titanium dioxide, zinc oxide, iron oxides, mica, MnO,
Fe₂O₃, Ce₂O₃, Al₂O₃, ZrO₂. (surface coatings: polymethyl methacrylate, methicone
(methylhydrogenpolysiloxane as described in CAS 9004-73-3), dimethicone, isopropyl
titanium triisostearate (as described in CAS 61417-49-0), metal soaps as magnesium
stearate (as described in CAS 4086-70-8), perfluoroalcohol phosphate as C9-15
fluoroalcohol phosphate (as described in CAS 74499-44-8; JP 5-86984, JP 4-330007)).
The primary particle size is an average of 15 nm-35 nm and the particle size in dispersion is
in the range of 100 nm-300 nm.
aminohydroxy-benzophenone derivatives disclosed in DE 10011317, EP 1133980 and EP 1046391
phenyl-benzimidazole derivatives as disclosed in EP 1167358
the UV absorbers described in “Sunscreens”, Eds. N. J. Lowe, N. A. Shaath, Marcel Dekker,
Inc., New York and Basle or in Cosmetics & Toiletries (107), 50ff (1992) also can be used
as additional UV protective substances.

The antimicrobial agents of the present invention are ingredients in a wide variety of cosmetic preparations. There come into consideration, for example, especially the following preparations like skin-care preparations, bath preparations, cosmetic personal care preparations, foot-care preparations; light-protective preparations, skin-tanning preparations, depigmenting preparations, insect-repellents, deodorants, antiperspirants, preparations for cleansing and caring for blemished skin, hair-removal preparations in chemical form (depilation), shaving preparations, fragrance preparations or cosmetic hair-treatment preparations.

The final formulations may exist in a wide variety of presentation forms, for example in the form of liquid preparations as a W/O, O/W, O/W/O, W/O/W or PIT emulsion and all kinds of microemulsions, in the form of a gel, an oil, a cream, milk or lotion, a powder, a lacquer, a tablet or make-up, a stick, a spray or an aerosol, a foam, or a paste.

The antimicrobial SiO_{z} flakes of the present invention show also antimicrobial activity against oral bacteria and exhibit an anti-plaque effectiveness, anti-gingivitis activities and help to reduce paradontitis.

Furthermore the oral composition may contain:

polishing agents, humectants, water, natural or synthetic thickener or gelling agent, alcohols, organic surface-active agents which can be cationic, anionic or non-ionic, flavoring agents, sweetening agents, agents used to diminish teeth sensitivity, whitening agents, preservatives, substances which release fluoride ions to protect against caries other agents such as chlorophyll compounds and/or ammoniated materials.

The antimicrobial SiO_{z} flakes of the present invention can also be used as additives in laundry detergent and/or fabric care compositions. The laundry detergent and/or fabric care compositions of the present invention preferably further comprise a detergent ingredient selected from cationic, anionic and/or nonionic surfactants and/or bleaching agent. The antimicrobial laundry detergent and/or fabric care compositions according to the invention can be liquid, paste, gels, bars, tablets, spray, foam, powder or granular forms. Granular compositions can also be in “compact” form, the liquid compositions can also be in a “concentrated” form.

The compositions of the invention may for example, be formulated as hand and machine laundry detergent compositions including laundry additive compositions and compositions suitable for use in the soaking and/or pretreatment of stained fabrics, rinse added fabric softener compositions. Pre-or post treatment of fabric include gel, spray and liquid fabric care compositions. A rinse cycle with or without the presence of softening agents is also contemplated.

When formulated as compositions suitable for use in a laundry machine washing method, the compositions of the invention preferably contain both a surfactant and a builder compound and additionally one or more detergent components preferably selected from organic polymeric compounds, bleaching agents, additional enzymes, suds suppressors, dispersants, lime-soap dispersants, soil suspension and anti-redeposition agents and corrosion inhibitors. Laundry compositions can also contain softening agents, as additional detergent components.

The laundry detergent and/or fabric care compositions of the present invention may also contain cationic fabric softening components which include the water-insoluble quaternary-ammonium fabric softening actives or the corresponding amine precursor, the most commonly used having been di-long alkyl chain ammonium chloride or methyl sulfate.

The laundry detergent and/or fabric care compositions of the present invention may also contain ampholytic, zwitterionic, and semi-polar surfactants.

In addition to the modified enzymes of the present invention, the laundry detergent and/or fabric care compositions may contain further one or more enzymes which provide cleaning performance, fabric care and/or sanitisation benefits.

The antimicrobial laundry detergent compositions according to the present invention may further comprise a builder system.

The antimicrobial laundry detergent and/or fabric care compositions herein may also optionally contain one or more iron and/or manganese chelating agents.

The compositions herein may also contain water-soluble methyl glycine diacetic acid (MGDA) salts (or acid form) as a chelant or co-builder useful with, for example, insoluble builders such as zeolites, layered silicates and the like.

Another optional ingredient is a suds suppressor, exemplified by silicones, and silica-silicone mixtures.

Other components such as soil-suspending agents, soil-release agents, optical brighteners, abrasives, bactericides, tarnish inhibitors, colouring agents, and/or encapsulated or non-encapsulated perfumes may be employed.

The laundry detergent and/or fabric care composition of the present invention can also contain dispersants:

The laundry detergent and/or fabric care compositions of the present invention can also include compounds for inhibiting dye transfer from one fabric to another of solubilized and suspended dyes encountered during fabric laundering operations involving colored fabrics.

Examples of antibacterial preparations (X =preferred combinations) of the Present invention:

A. Personal Care Compositions
O/W systems:

| Ingredients | | | | | | | | |
|---|---|---|---|---|---|---|---|---|
| Emulsifiers | 1 | 2 | 3 | 4 | 5 | 6 | 7 | 8 |
| Potassium Cetyl Phosphate 2%-5% | X | | | | | | | |
| Cetearyl Alcohol/Dicetyl Phosphate/Ceteth-10 Phosphate 2%-6% | | X | | | | | | |
| Sodium Stearyl Phtalamate 1%-2% | | | X | | | | | |
| Cetearyl Alcohol/Behentrimonium Methosulfate 1%-5% | | | | X | | | | |
| Quaternium-32 1%-5% | | | | | X | | | |
| Dimethicone copolyol/Caprylic/Capric Triglyceride (1%-4%) | | | | | | X | | |
| Steareth-2/Steareth-21 2%-5% | | | | | | | X | |
| Polyglyceryl Methyl Glucose Distearate 1%-4% | | | | | | | | X |
| Lipophilic emollient/dispersant oil 15%-20% | X | X | X | X | X | X | X | X |
| Fatty Alcohols and/or Waxes 1%-5% | X | X | X | X | X | X | X | X |
| Thickeners (water swellable thickeners) 0.5%-1.5% | X | X | X | X | X | X | X | X |
| Preservatives 0.5%-1% | X | X | X | X | X | X | X | X |
| Chelating agents (such as EDTA) 0%-0.2% | X | X | X | X | X | X | X | X |
| Antioxidants 0.05%-0.2% | X | X | X | X | X | X | X | X |
| Water deionized Qs 100% | X | X | X | X | X | X | X | X |
| Perfume oils 0.1%-0.4% | X | X | X | X | X | X | X | X |
| Antimicrobial SiO_{z} flakes 0.1%-20% | X | X | X | X | X | X | X | X |

W/O systems

| Ingredients | 1 | 2 | 3 | 4 | 5 |
|---|---|---|---|---|---|
| Emulsifiers | X | X | X | X | X |
| Polyglyceryl-2 Dipolyhydroxystearate 2%-4% | X | X | X | X | X |
| PEG-30 Dipolyhydroxystearate 2%-4% | | X | | | |
| Rapeseed Oil Sorbitol Esters 1%-5% | | | X | | |
| PEG-45/Dodecyl Glycol Copolymer 1%-5% | | | | X | |
| Sorbitan Oleate/Polycerol-3 ricinoleate 1%-5% | | | | | X |
| Lipophilic emollient/dispersant oil 10%-20% | X | X | X | X | X |
| Fatty Alcohols and/or Waxes 10%-15% | X | X | X | X | X |
| Electrolytes (NaCl, MgSO₄) 0.5%-1% | X | X | X | X | X |
| Polyol phase (Propylene glycol, glycerin) 1%-8% | X | X | X | X | X |
| Preservatives 0.3%-0.8% | X | X | X | X | X |
| Perfume oils 0.1%-0.4% | X | X | X | X | X |
| Chelating agents (such as EDTA) 0%-0.2% | X | X | X | X | X |
| Antioxidants 0.05%-0.2% | X | X | X | X | X |
| Water deionized Qs 100% | X | X | X | X | X |
| Antimicrobial SiO_{z} flakes 0.1%-20%. | X | X | X | X | X |

Multiple emulsions

| Ingredients | 1 | 2 | 3 | 4 | 5 | 6 | 7 | 8 | 9 | 10 | 11 | 12 |
|---|---|---|---|---|---|---|---|---|---|---|---|---|
| PEG-30 Dipolyhydroxystearate | X | | | | | | | | | X | | X |
| (2%-6%) | | | | | | | | | | | | |
| Cetyl Dimethicone Copolyol | | X | | | | | | | X | | | |
| 1%-3% | | | | | | | | | | | | |
| PEG-30 Dipolyhydroxystearate/ | | | X | | | | | X | | | | |
| Steareth-2/Steareth-21 4%-6% | | | | | | | | | | | | |
| Polyglyceryl-2 Dipolyhydroxy- | | | | X | | | X | | | | | |
| stearate 1%-3% | | | | | | | | | | | | |
| Polyglyceryl-6 Ricinoleate 1%-3% | | | | | X | X | | | | | X | |
| Oil phase 15%-30% | | | | | | | | | | | | |
| Fatty acid esters | X | X | X | X | X | | | | | | X | X |
| Natural and synthetic | | | | | | X | X | X | X | X | X | X |
| Triglycerides | | | | | | | | | | | | |
| Hydrocarbon oils | X | X | X | X | X | | | | | | X | X |
| Silicone oils | | | | | | X | X | X | X | X | X | X |
| Preservatives 0.3%-0.8% | X | X | X | X | X | X | X | X | X | X | X | X |
| Water Deioniz. qs 100% | X | X | X | X | X | X | X | X | X | X | X | X |
| Sorbitan Stearate/Sucrose | X | | | | | | | X | | | | X |
| Cocoate 3%-7% | | | | | | | | | | | | |
| Sucrose Laurate 3%-7% | | X | | | | | X | | | | X | |
| Poloxamer 407 3%-7% | | | X | | | X | | | X | | | |
| Polyoxyethylene(20)Sorbate | | | | X | X | | | | | X | | |
| Monoleate 3%-5% | | | | | | | | | | | | |
| Primary emulsion W1/O 50% | X | X | X | X | X | X | X | X | X | X | X | X |
| Thickeners (water swellable | X | X | X | X | X | X | X | X | X | X | X | X |
| polymers) 0.3%-1% | | | | | | | | | | | | |
| Water deionized Qs 100% | X | X | X | X | X | X | X | X | X | X | X | X |
| Perfume oils 0.1%-0.4% | X | X | X | X | X | X | X | X | X | X | X | X |
| Antimicrobial SiO_{z} | X | X | X | X | X | X | X | X | X | X | X | X |
| flakes 0.1%-20% | | | | | | | | | | | | |

O1/W/O2 emulsions

| Ingredients | 1 | 2 | 3 | 4 | 5 | 6 | 7 | 8 |
|---|---|---|---|---|---|---|---|---|
| Primary emulsion O1/W | | | | | | | | |
| PEG-60 Hydrogenated Castor Oil 25% | X | | | X | X | | | X |
| Steareth-25 25% | | X | X | | | X | X | |
| Oil phase 75% | | | | | | | | |
| Fatty acid esters | X | | X | | | | | |
| Natural and synthetic Triglycerides | | X | | X | | | | |
| Hydrocarbon oils | | | | | X | | X | |
| Silicone oils | | | | | | X | | X |
| Preservatives 0.3%-0.8% | X | X | X | X | X | X | X | X |
| Water deionized Qs 100% | X | X | X | X | X | X | X | X |
| Non ionic multifunctional W/O emulsifier 2%-5% | X | X | X | X | X | X | X | X |
| Waxes 1%-5% | X | X | X | X | X | X | X | X |
| Oil phase 20%-30% | X | X | X | X | X | X | X | X |
| Silicone oils | | | | | | | | |
| Primary emulsion O1/W 15% | X | X | X | X | X | X | X | X |
| Electrolytes (NaCl, MgSO₄) 0.1%-0.5% | X | X | X | X | X | X | X | X |
| Water deionized Qs 100% | X | X | X | X | X | X | X | X |
| Perfume oils 0.1%-0.4% | X | X | X | X | X | X | X | X |
| Antimicrobial SiO_{z} flakes 0.1%-20% | X | X | X | X | X | X | X | X |

Microemulsions

| Ingredients | 1 | 2 | 3 | 4 | 5 | 6 | 7 | 8 | 9 | 10 |
|---|---|---|---|---|---|---|---|---|---|---|
| PEG-8 Caprylic/Capric Glycerides 10%-25% | X | | | X | X | | | X | X | |
| PPG-5-ceteth-20 10%-25% | | X | X | | | X | X | | | X |
| Polyglyceryl-6 Isostearate 5%-15% | X | | X | | | | | | | |
| Polyglyceryl-3 Diisostearate 5%-15% | | X | | X | | | | | | |
| Polyglyceryl-6 Dioleate 5%-15% | | | | | X | | X | | | |
| PPG-10 Cetyl Ether 5%-15% | | | | | | X | | X | | |
| Ethoxydiglycol 5%-15% | | | | | | | | | X | X |
| Oil phase 10%-80% | X | X | X | X | X | X | X | X | X | X |
| Isostearyl Benzoate | X | X | X | X | X | X | X | X | X | X |
| Isostearyl Isostearate | X | X | X | X | X | X | X | X | X | X |
| PEG-7 Glyceryl Cocoate | X | X | X | X | X | X | X | X | X | X |
| Cyclomethicone | X | X | X | X | X | X | X | X | X | X |
| Polyalcohols/Humectants 1%-10% | X | X | X | X | X | X | X | X | X | X |
| Preservatives 0.3-0.8% | X | X | X | X | X | X | X | X | X | X |
| Perfume oils 0.1%-0.4% | X | X | X | X | X | X | X | X | X | X |
| Water Deioniz. qs 100% | X | X | X | X | X | X | X | X | X | X |
| Antimicrobial SiO_{z} | X | X | X | X | X | X | X | X | X | X |
| flakes 0.1%-20% | | | | | | | | | | |
| UV-absorber as described in | X | X | X | X | X | X | X | X | X | X |
| table 1-3 0%-30% | | | | | | | | | | |

G - Aqueous

| Ingredients | | | | | | | | | | | | |
|---|---|---|---|---|---|---|---|---|---|---|---|---|
| Thickeners | 1 | 2 | 3 | 4 | 5 | 6 | 7 | 8 | 9 | 10 | 11 | 12 |
| Natural Thickener 1%-5% | X | | | | | X | X | | | | | X |
| Semi-synthetic Thickener 1%-5% | | X | | | X | | | X | | | X | |
| Synthetic Thickener 0.3%-1.3% | | | X | X | | | | | X | X | | |
| Neutralizing Agents 0.5%-1.5% | X | X | X | X | X | X | X | X | X | X | X | X |
| Polyols - Humectants 5%-50% | X | X | X | X | X | X | X | X | X | X | X | X |
| Polyquaternium series 1%-5% | X | X | X | | | | X | X | X | | | |
| PVM/MA Copolymer 1%-5% | | | | X | X | X | | | | X | X | X |
| Preservatives 0.5%-1% | X | X | X | X | X | X | X | X | X | X | X | X |
| Chelating Agents (as EDTA) <0.1% | X | X | X | X | X | X | X | X | X | X | X | X |
| Water Deioniz. qs 100% | X | X | X | X | X | X | X | X | X | X | X | X |
| Perfume oils 0.05%-0.4% | X | X | X | X | X | X | X | X | X | X | X | X |
| Ethoxylated Glyceryl ethers 0.1%-5% | X | X | X | | | | | | | | | |
| Polysorbates 0.1%-5% | | | | X | X | X | | | | | | |
| Ethoxylated Oleyl ethers 0.1%-5% | | | | | | | X | X | X | X | X | X |
| Antimicrobial SiO_{z} flakes 0.1%-20% | X | X | X | X | X | X | X | X | X | X | X | X |

Oleogels

| Ingredients | 1 | 2 | 3 | 4 | 5 | 6 | 7 | 8 | 9 | 10 |
|---|---|---|---|---|---|---|---|---|---|---|
| Hydrogenated Lecithin 1%-10% | X | | | | | | | | | X |
| Silica Dimethyl Silylate 1%-10% | | X | | | | | | | X | |
| Silica 1%-5% | | | X | | | | | X | | |
| C₂₄₋₂₈ Alkyl Dimethicone 1%-5% | | | | X | | | X | | | |
| Aluminium or Magnesium Stearate 1%-5% | | | | | X | X | | | | |
| Polyols - Humectants 5%-70% | X | X | X | X | X | X | X | X | X | X |
| Oil phase 20%-90% | | | | | | | | | | |
| Dicaprylyl Ether | X | | | | | X | | X | | |
| Phenyl Trimethicone | | X | | | | | X | | | |
| Hydrogenated Polyisobutene | | | X | | | | | | | |
| Isopropyl Isostearate | | | | X | | | | | X | |
| Oleogel basis (Mineral oil and hydrogenated | | | | | X | | | | | X |
| Butylene/Ethylene or Ethylene/Propylene | | | | | | | | | | |
| Styrene Copolymer) | | | | | | | | | | |
| Silicone wax 1%-10% | X | X | X | X | X | X | X | X | X | X |
| Dimethiconol Behenate | X | X | X | X | X | X | X | X | X | X |
| Dimethiconol Stearate | X | X | X | X | X | X | X | X | X | X |
| Perfume oils 0.1%-0.5% | X | X | X | X | X | X | X | X | X | X |
| Antioxidants 0.05%-0.2% | X | X | X | X | X | X | X | X | X | X |
| Antimicrobial SiO_{z} flakes 0.1%-20% | X | X | X | X | X | X | X | X | X | X |

Light/dry cosmetic oils

| Ingredients | 1 | 2 | 3 | 4 |
|---|---|---|---|---|
| Hydrocarbon oils 30%-70% | X | | | X |
| Fatty acid esters branched or not 10%-50% | | X | X | |
| Silicones/Siloxanes 0%-10% | X | | X | |
| Perfluorinated oils and Perfluoroethers 0%-10% | | X | | X |
| Viscosifying agents 0%-10% | X | X | X | X |
| Esters of long chain acids and alcohols 0%-2% | X | X | X | X |
| Antioxidants 0.1%-1% | X | X | X | X |
| Solubilisants/dispersing agents 0%-5% | X | X | X | X |
| Perfume oils 0.1%-0.5% | X | X | X | X |
| Antimicrobial SiO_{z} flakes 0.1%-20%. | X | X | X | X |

Foaming/mousse products

| Ingredients | 1 |
|---|---|
| SD Alcohol 40 0%-8% | X |
| Propellant 8%-15% | X |
| Nonionic Emulsifier/Surfactant 0.5%-3% | X |
| Corrosion Inhibitor 0%-1% | X |
| Perfume oils 0.1%-0.5% | X |
| Preservatives 0.1%-1% | X |
| Miscellaneous 0%-1% | X |
| Antimicrobial SiO_{z} flakes 0.1%-20%. | X |

Stick products

| Ingredients | 1 |
|---|---|
| Waxes 15%-30% | X |
| Natural and silicone oils 20%-75% | X |
| Lanoline derivatives 5%->50% | X |
| Esters of lanolin | x |
| Acetylated lanolin | x |
| Lanolin oil | x |
| Colorants and pigments 10%-15% | X |
| Antioxidants 0.1%-0.8% | X |
| Perfume oils 0.1%-2% | X |
| Preservatives 0.1%-0.7% | X |
| Antimicrobial SiO_{z} flakes 0.1%-20% | X |

Liquid and compact

| Ingredients | 1 | 2 |
|---|---|---|
| Liquid foundation | | |
| Powder phase 10%-15% | X | |
| Oil phase 30%-40%; 75% (only for anhydrous form) | X | |
| Thickener/suspending agents 1%-5% | X | |
| Film forming polymers 1%-2% | X | |
| Antioxidants 0.1%-1% | X | |
| Perfume oils 0.1%-0.5% | X | |
| Preservatives 0.1%-0.8% | X | |
| Water deionized Qs 100% | X | |
| Compact powder | | |
| Powder phase 15%-50% | | X |
| Oil phase 15%-50% | | X |
| Polyol phase 5%-15% | | X |
| Antioxidants 0.1%-1% | | X |
| Perfume oils 0.1%-0.5% | | X |
| Preservatives 0.1%-0.8% | | X |
| For the two product forms | | |
| Antimicrobial SiO_{z} flakes 0.1%-20% | X | X |

Conditioning Shampoos

| Ingredients | 1 |
|---|---|
| Primary surfactants (listed previously) 5%-10% | X |
| Secondary surfactants (listed previously) 5%-15% | X |
| Foam Stabilizers (listed previously) 0%-5% | X |
| Water deionized 40%-70% | X |
| Actives 0-10% | X |
| Conditioners | x |
| Refatting agents | x |
| Moisturizing agents | x |
| Thickeners/Rheology mofifiers 0%-3% | X |
| Humectants 0%-2% | X |
| PH adjusting agents 0%-1% | X |
| Preservatives 0.05%-1% | X |
| Perfume oils 0.1%-1% | X |
| Antioxidants 0.05%-0.20% | X |
| Chelating Agents (EDTA) 0%-0.2% | X |
| Opascifying agents 0%-2% | X |
| Antimicrobial SiO_{z} flakes 0.1%-20% | X |

Antimicrobial Cleansing Compositions

| | Ex. | Ex. | Ex. | Ex. | Ex. | Ex. | Ex. | Ex. | Ex. | Ex. |
|---|---|---|---|---|---|---|---|---|---|---|
| Component | 1 | 2 | 3 | 4 | 5 | 6 | 7 | 8 | 9 | 10 |
| Mineral oil | 1.00 | 1.00 | 1.00 | 1.00 | — | — | — | 1.00 | 1.00 | 1.00 |
| Propylene glycol | 1.00 | 1.00 | 1.00 | 1.00 | 1.00 | 1.00 | 1.00 | 1.00 | 1.00 | 1.00 |
| Ammonium | 0.60 | 0.60 | 0.60 | 0.60 | 0.60 | 0.60 | 0.60 | 0.60 | 0.60 | 0.60 |
| Lauryl Sulfate | | | | | | | | | | |
| Citric Acid | 4.00 | — | — | — | — | — | — | 2.50 | 2.50 | 4.00 |
| Sodium Citrate | 3.30 | — | 2.00 | — | — | — | 3.70 | 2.00 | 2.00 | 3.20 |
| Succinic Acid | — | 4.00 | — | — | 4.00 | 4.00 | — | — | — | — |
| Sodium Succinate | — | 3.30 | 0.00 | 0.00 | 3.20 | 3.00 | — | — | — | — |
| Malic Acid | — | — | — | 4.00 | — | — | 4.00 | — | — | — |
| Sodium Malonate | — | — | — | 3.20 | — | — | — | — | — | — |
| Steareth 20 | 0.55 | 0.55 | 0.55 | 0.55 | — | 0.55 | — | — | 0.08 | 0.28 |
| Steareth 2 | 0.45 | 0.45 | 0.45 | 0.45 | — | 0.45 | — | 0.45 | 0.07 | 0.23 |
| Oleth 20 | — | — | — | — | — | — | — | — | 0.08 | 0.28 |
| Oleth 2 | — | — | — | — | — | — | — | — | 0.07 | 0.23 |
| Antimicrobial | 0.15 | 0.15 | 0.15 | 0.15 | 0.15 | 0.01 | 0.50 | 0.50 | 0.15 | 0.25 |
| SiO_{z} flakes | | | | | | | | | | |
| Thymol | — | — | — | — | — | 1.00 | — | — | — | — |
| Miscellaneous | 0.36 | 0.36 | 0.36 | 0.36 | 0.36 | 0.36 | 0.36 | 0.36 | 0.36 | 0.36 |
| Water | q.s. | q.s. | q.s. | q.s. | q.s. | q.s. | q.s. | q.s. | q.s. | q.s. |
| pH | 4.0 | 4.5 | 3.9 | 3.9 | 3.9 | 3.9 | 3.9 | 3.9 | 3.9 | 3.9 |

Antimicrobial Cleansing Compositions

| Component | Ex. 11 | Ex. 12 | Ex. 13 | Ex. 14 | Ex. 15 |
|---|---|---|---|---|---|
| Mineral oil | 1.00 | 1.00 | 1.00 | 1.00 | — |
| Propylene glycol | 1.00 | 1.00 | 1.00 | 1.00 | 1.00 |
| Ammonium Lauryl Sulfate | — | — | — | — | 0.60 |
| Ammonium Laureth Sulfate | — | 5.00 | — | — | — |
| Hostapur SAS 60 (SPS) | 1.00 | — | — | — | — |
| C₁₄₋₁₆ Sodium-Olefin | — | — | 2.00 | — | — |
| Sulfonate | | | | | |
| Sodium Lauroyl Sarcosinate | — | — | — | 1.00 | — |
| Citric Acid | 0.055 | 7.50 | — | — | — |
| Sodium Citrate | — | 4.00 | 2.00 | — | — |
| Succinic Acid | 4.00 | — | — | — | — |
| Sodium Succinate | 0.67 | — | — | — | — |
| Malonic Acid | — | — | — | 4.00 | — |
| Malic Acid | — | — | 2.50 | — | — |
| Sodium Malonate | — | — | — | 3.20 | — |
| Salicylic Acid | — | — | — | — | 0.50 |
| Steareth 20 | 0.55 | 0.55 | 0.55 | 0.55 | 0.55 |
| Steareth 2 | 0.45 | 0.45 | 0.45 | 0.45 | 0.45 |
| Antimicrobial SiO_{z} flakes | 0.15 | 3.00 | 0.15 | 0.01 | 0.15 |
| Cocamidopropyl Betaine | — | — | — | 4.00 | — |
| Polyquat 10 | — | — | — | 0.40 | — |
| Miscellaneous | 0.36 | 0.36 | 0.36 | 0.36 | 0.36 |
| Water | q.s. | q.s. | q.s. | q.s. | q.s. |
| ph | 3-6 | 3-6 | 3 | 6 | 3 |

| | Formulation | | | | |
|---|---|---|---|---|---|
| | 1 | 2 | 3 | 4 | 5 |
| Antimicrobial SiO_{z} flakes | 0.6 | 0.6 | 0.6 | 0.6 | 0.6 |
| sodium dodecylbenzenesulfonate | 6 | 6 | 6 | 6 | 6 |
| sodium lauryl sulfate | 8 | 8 | 8 | 8 | 8 |
| Pareth 45-7 (Dobanol 45-7) | 4 | 4 | 4 | 4 | 4 |
| ethanol | 9 | 9 | 9 | 9 | 9 |
| sodium cumenesulfonate | 5 | — | 5 | 5 | — |
| soap noodles (Mettler) | 5 | 7 | 7 | 5 | 7 |
| trisodium citrate dihydrate | 2 | 2 | 2 | 2 | 2 |
| triethanolamine | 5 | 5 | 5 | 5 | 5 |
| fluorescent whitening agents | 0.3 | 0.3 | 0.3 | 0.3 | 0.3 |
| water to | 100 | 100 | 100 | 100 | 100 |

B. Home and Fabric Care Formulations

| | Formulation | | | | | | | | | | |
|---|---|---|---|---|---|---|---|---|---|---|---|
| Components | 1 | 2 | 3 | 4 | 5 | 6 | 7 | 8 | 9 | 10 | 11 |
| Antimicrobial SiO_{z} flakes | 0.9 | 0.9 | 0.9 | 0.9 | 0.9 | 0.9 | 0.9 | 0.9 | 0.9 | 0.9 | 0.9 |
| dodecylbenzenesulfonic acid | 7.5 | | | | | | | 8.5 | | | |
| sodium dodecylbenzenesulfonate | | 27 | 23.6 | 10 | 28 | | | | 20 | 24 | 6 |
| sodium laureth sulfate 3 EO | 17 | 10 | | | | | | | | | |
| sodium lauryl sulfate | | | | | | 6 | | | | | 8 |
| coconut acid | 12.5 | | | 10 | 4 | 4 | 10 | | | 10 | |
| C₁₂₋₁₃ Pareth-7 | 10 | | | | | | | 26.9 | 27.8 | 25 | 4 |
| PEG-7 C₁₃ oxoalcohol | | | | 20 | 9 | 14.5 | 12 | 29 | 26 | | |
| PEG-8 C₁₃₋₁₅ fatty alcohol | | | | | | | 10 | | | | |
| alkyl polyglucoside | | | 5 | | | 1 | 2 | | | | |
| laureth-10 | | 5 | | | | | | | | | |
| PPG | | | | 2 | 3 | 8 | | | | | |
| sodium carbonate | | | 2 | | | | | | | | |
| sodium tripolyphosphate | | | 20 | | | | | | | | |
| potassium tripolyphosphate 50% | | 22 | | | | | | | | | |
| sodium cumenesulfonate 40% | | 25 | | | | | | | | | |
| trisodium citrate | 5.5 | | | | | 2 | | | | | 2 |
| lauryltrimonium chloride | 0.7 | | | | | | | | | | |
| polycarboxylate | | | | 13 | 18 | 15 | 10 | 23 | 16.2 | | |
| 2-propanol | 6 | | | 7 | 3 | | 4 | 9.5 | 8 | | |
| ethanol | 6 | | | | | | | | | | 9 |
| glycerol | | | | | | | | | | 20 | |
| propylene glycol | | | | | | | | | | 6 | |
| NaOH | 3.2 | | | 2 | 1 | 2.3 | 1.8 | 1.1 | | 1.8 | 4 |
| fluorescent whitening agent | 0.1 | 0.1 | 0.1 | 0.1 | 0.1 | 0.1 | 0.1 | | | | 0.1 |
| Tinopal CBS-x | | | | | | | | | | | |
| fluorescent whitening agent | | | | | | | | 0.1 | 0.1 | 0.1 | |
| Tinopal CBS-CL | | | | | | | | | | | |
| Soap | | | | | | | | | | | 7 |
| water to | 100 | 100 | 100 | 100 | 100 | 100 | 100 | 100 | 100 | 100 | 100 |

| | formulation | | |
|---|---|---|---|
| components | 13a | 13b | 13c |
| Antimicrobial SiO_{z} flakes | 0.9 | 0.9 | 0.45 |
| sodium laureth sulfate | 1.2 | | |
| cocamidopropyl betaine | 1 | | |
| lauramine oxide | 1 | | |
| sodium Citrate | 4 | | |
| sodium carbonate | 3 | | |
| ethanol | 3 | | |
| sodium C₁₄₋₁₇ alkyl sec. Sulfonate | | 16.6 | |
| sodium laurylsulfate | | 20 | |
| Laureth-09 | | 3 | |
| sodium cumolsulfonate | | 5 | |
| sodium chloride | | 3 | |
| Quaternium 18 and iospropylalcohol | | | 4 |
| Pareth-25-7 | | | 0.5 |
| water to | 100 | 100 | 100 |

Liquid Washing Formulation

| | Formulation | | | | |
|---|---|---|---|---|---|
| | 1 | 2 | 3 | 4 | 5 |
| Antimicrobial SiO_{z} flakes | 0.6 | 0.6 | 0.6 | 0.6 | 0.6 |
| sodium dodecylbenzenesulfonate | 6 | 6 | 6 | 6 | 6 |
| sodium lauryl sulfate | 8 | 8 | 8 | 8 | 8 |
| Pareth 45-7 (Dobanol 45-7) | 4 | 4 | 4 | 4 | 4 |
| ethanol | 9 | 9 | 9 | 9 | 9 |
| sodium cumenesulfonate | 5 | — | 5 | 5 | — |
| soap noodles (Mettler) | 5 | 7 | 7 | 5 | 7 |
| trisodium citrate dihydrate | 2 | 2 | 2 | 2 | 2 |
| triethanolamine | 5 | 5 | 5 | 5 | 5 |
| fluorescent whitening agents | 0.3 | 0.3 | 0.3 | 0.3 | 0.3 |
| water to | 100 | 100 | 100 | 100 | 100 |

Liquid Washing Formulation

| | Formulation | | | | | | | | | | |
|---|---|---|---|---|---|---|---|---|---|---|---|
| Components | 1 | 2 | 3c | 4 | 5 | 6 | 7 | 8 | 9 | 10 | 11 |
| Antimicrobial SiO_{z} flakes | 0.5 | 1.0 | 0.5 | 0.2 | 0.9 | 0.6 | 1.5 | 2 | 0.5 | 0.1 | 0.2 |
| dodecylbenzenesulfonic acid | 7.5 | | | | | | | 8.5 | | | |
| sodium dodecylbenzenesulfonate | | 27 | 23.6 | 10 | 28 | | | | 20 | 24 | 6 |
| sodium laureth sulfate 3 EO | 17 | 10 | | | | | | | | | |
| sodium lauryl sulfate | | | | | | 6 | | | | | 8 |
| coconut acid | 12.5 | | | 10 | 4 | 4 | 10 | | | 10 | |
| C₁₂₋₁₃ Pareth-7 | 10 | | | | | | | 26.9 | 27.8 | 25 | 4 |
| PEG-7 C₁₃ oxoalcohol | | | | 20 | 9 | 14.5 | 12 | 29 | 26 | | |
| PEG-8 C₁₃₋₁₆ fatty alcohol | | | | | | | 10 | | | | |
| alkyl polyglucoside | | | 5 | | | 1 | 2 | | | | |
| laureth-10 | | 5 | | | | | | | | | |
| PPG | | | | 2 | 3 | 8 | | | | | |
| sodium carbonate | | | 2 | | | | | | | | |
| sodium tripolyphosphate | | | 20 | | | | | | | | |
| potassium tripolyphosphate 50% | | 22 | | | | | | | | | |
| sodium cumenesulfonate 40% | | 25 | | | | | | | | | |
| trisodium citrate | 5.5 | | | | | 2 | | | | | 2 |
| lauryltrimonium chloride | 0.7 | | | | | | | | | | |
| polycarboxylate | | | | 13 | 18 | 15 | 10 | 23 | 16.2 | | |
| 2-propanol | 6 | | | 7 | 3 | | 4 | 9.5 | 8 | | |
| ethanol | 6 | | | | | | | | | | 9 |
| glycerol | | | | | | | | | | 20 | |
| propylene glycol | | | | | | | | | | 6 | |
| NaOH | 3.2 | | | 2 | 1 | 2.3 | 1.8 | 1.1 | | 1.8 | 4 |
| fluorescent whitening agent | 0.1 | 0.1 | 0.1 | 0.1 | 0.1 | 0.1 | 0.1 | | | | 0.1 |
| Tinopal CBS-x | | | | | | | | | | | |
| fluorescent whitening agent | | | | | | | | 0.1 | 0.1 | 0.1 | |
| Tinopal CBS-CL | | | | | | | | | | | |
| Soap | | | | | | | | | | | 7 |
| water to | 100 | 100 | 100 | 100 | 100 | 100 | 100 | 100 | 100 | 100 | |

Liquid Washing Formulation

| | formulation | | |
|---|---|---|---|
| components | 13a | 13b | 13c |
| Antimicrobial SiO_{z} flakes | 0.5 | 1.0 | 0.2 |
| sodium laureth sulfate | 1.2 | | |
| cocamidopropyl betaine | 1 | | |
| lauramine oxide | 1 | | |
| sodium Citrate | 4 | | |
| sodium carbonate | 3 | | |
| ethanol | 3 | | |
| sodium C₁₄₋₁₇ alkyl sec. Sulfonate | | 16.6 | |
| sodium laurylsulfate | | 20 | |
| Laureth-09 | | 3 | |
| sodium cumolsulfonate | | 5 | |
| sodium chloride | | 3 | |
| Quaternium 18 and iospropylalcohol | | | 4 |
| Pareth-25-7 | | | 0.5 |
| water to | 100 | 100 | 100 |

The antimicrobial SiO_{z} flakes of the present invention can also be used for the production of antimicrobial chewing gums (U.S. Pat. No. 6,365,130).

Accordingly, the present invention also relates to an antimicrobial chewing gum comprising:

(a) a chewing gum base and

(b) the antimicrobial SiO_{z} flakes of the present invention, wherein the antimicrobial SiO_{z} flakes are present in an amount of from about 0.05 to 50 weight percent, based on the weight of the chewing gum composition.

Fiber materials which can be treated with the antimicrobial SiO_{z} flakes of the present invention are materials comprising for example, silk, leather, wool, polyamide, for example nylon (including nylon-6, Nylon-66), or polyurethanes, polyester, polyacrylonitrile polypropylene, polyethylene and cellulose-containing fiber materials of all kinds, for example natural cellulose fibers, such as cotton, linen, jute and hemp, and also viscose staple fiber and regenerated cellulose.

Polyester fiber materials which can be treated with the antimicrobial SiO_{z} flakes of the present invention will be understood as including cellulose ester fibers such as cellulose secondary acetate and cellulose triacetate fibers and, preferably, linear polyester fibers which may also be acid-modified, and which are obtained by the condensation of terephthalic acid with ethylene glycol or of isophthalic acid or terephthalic acid with 1,4-bis(hydroxy-methyl)cyclohexane, as well as copolymers of terephthalic and isophthalic acid and ethylene glycol. The linear polyester fiber material (PES) hitherto used almost exclusively in the textile industry consists of terephthalic acid and ethylene glycol.

The fiber materials may also be used as blends of natural fibers like cotton, wool or jute with each other or with synthetic fiber materials like PES, Nylon or polypropylene or blends of synthetic fiber materials with each other. Typical fiber blends are of polyacrylonitrile-polyester, polyamide/polyester, polyester/cotton, polyester/viscose and polyester/wool.

The textile fiber material can be in different forms of presentation, preferably as woven or knitted fabrics or as piece goods such as knitgoods, woven fabrics nonwoven textiles, carpets, piece garments also as yarn on cheeses, warp beams and the like or finished goods in any other form, preferably T-shirts, sport wears, running bra, sweaters, coats, lingeries, underwears and socks.

The fibers or fiber blends can be treated batchwise or continuously.

In continuous treatment methods, the treatment liquors, which may optionally contain assistants, are applied to yarns, fabric, piece goods, for example, by padding or slop-padding and are developed by thermofixation or HT steaming processes.

The fiber material, which is treated by the present process is characterized by having an essentially homogeneous distribution of the antimicrobial SiO_{z} flakes throughout the fiber cross-section.

The process according to the invention is carried out in accordance with known textile dyeing and printing processes using conventional pigments as described, for example, in Textile Chemist and Colorist 25 (1993) 31-37.

The antimicrobial SiO_{z} flakes of the present invention are advantageously used in the dyeing preparations, for example dye baths or printing pastes, in dispersed form.

During dispersion of the antimicrobial SiO_{z} flakes of the present invention and during processing thereof, conditions under which only relatively weak shearing forces occur are preferably maintained so that the antimicrobial SiO_{z} flakes of the present invention will not be broken up into smaller fragments.

The customary dispersants, preferably non-ionic dispersants, can be used for the preparation of the dispersions.

Suitable binders for the process according to the invention include the pigment dyestuff binders customarily employed in textile dyeing and textile printing, for example acrylate-based, urethane-based or butadiene-based binders. Such binders are known to the person skilled in the art.

Suitable acrylate binders are, for example, acrylic polymers, such as, for example, poly(meth)acrylates, or mixed polymers of (meth)acrylates with suitable comonomers, such as, for example, acrylic, methacrylic, maleic, fumaric, itaconic, mesaconic, citraconic, vinylacetic, vinyloxyacetic, vinylpropionic, crotonic, aconitic, allylacetic, allyloxyacetic, allylmalonic, 2-acrylamido-2-methylpropanesufonic, glutaconic or allylsuccinic acid, or with esters of those acids, (meth)acrylamide, N-vinylpyrrolidone, N-vinylformamide, N-vinylacetamide, (meth)acrolein, N-vinyl-N-methylacetamide, vinylcaprolactam, styrene derivatives or vinylphosphonic acid; polyamide derivatives; synthetic resin dispersions; vinyl-based mixed polymers; diamide/aldehyde precondensates; mixed polymers comprising N-vinyllactam or butadiene-based polymers. Suitable acrylate binders are soluble in aqueous medium or in aqueous medium containing water-miscible organic solvents, where applicable with the addition of bases. The said acrylate binders are preferably used in the form of an aqueous formulation. Such acrylate binders are commercially available in acidic form or in partially or completely neutralised form, for example Primal® (Rohm & Haas), Neocryl® (NeoResins), Carbocet® (BF Goodrich), Joncryl® (Johnson Polymers) or ALCOPRINT®, or KNITTEX® (Ciba Specialty Chemicals) binders.

According to an embodiment of the present invention, the dyeing preparation, for example the printing paste or the dye bath, is prepared by using a concentrated formulation comprising the antimicrobial SiO_{z} flakes of the present invention and the binder. Such formulations will preferably be aqueous formulations. The weight ratio between the antimicrobial SiO_{z} flakes and binder is preferably from 1:1 to 1:50, especially from 1:1 to 1:10. A weight ratio of from 1:1 to 1:5 is especially preferred. The antimicrobial SiO_{z} flakes of the present invention are present in the formulation preferably in an amount of from 2 to 80 g/kg, especially in an amount of from 5 to 50 g/kg. The binder is present in the formulation preferably in an amount of from 20 to 200 g/kg, especially in an amount of from 30 to 150 g/kg.

The dyeing preparations may additionally comprise further auxiliaries customarily used, for example, in pigment printing, for example crosslinkers.

Suitable crosslinkers are, for example, water-soluble melamine, formaldehyde/melamine and formaldehyde/urea resins or precondensates, such as trimethylolmelamine, hexamethylolmelamine or dimethylol urea, or water-soluble formaldehyde (pre)condensation products with formamide, thiourea, guanidine, cyanamide, dicyandiamide and/or water-soluble organic sulfonates, such as, for example, the sodium salt of naphthalenesulfonic acid, or glyoxalic urea derivatives, such as, for example, the compound of formula 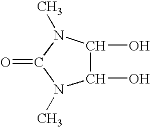
and especially N-methylol derivatives of nitrogen-containing compounds, such as, for example, non-etherified or etherified melamine/formaldehyde condensation products or N-methylol urea compounds.

Examples of non-etherified or etherified melamine/formaldehyde condensation products are the compounds of formulae

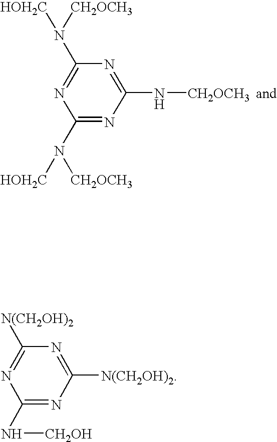

The non-etherified or etherified N-methylol urea compounds are, for example, reaction products of formaldehyde with urea or urea derivatives, which reaction products may have been subsequently etherified, suitable urea derivatives being, for example, cyclic ethylene or propylene ureas that may also contain substituents such as hydroxyl groups in the alkylene group, urones or unsubstituted or substituted triazone resins.

Examples of corresponding N-methylol urea compounds are unmodified or modified N-methylolhydroxyethylene urea products, for example the compounds of formula 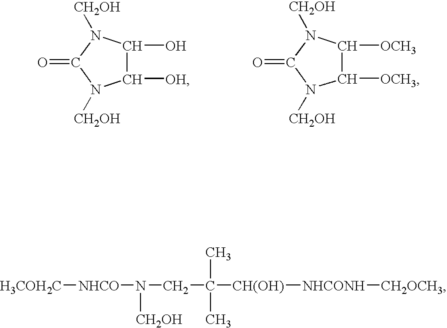
based on propylene urea or ethylene urea/melamine.

Preferred crosslinkers are unmodified or modified N-methylolhydroxyethylene urea compounds, methylolation products based on propylene urea or ethylene urea/melamine and, especially, non-etherified or etherified melamine/formaldehyde condensation products. It is also possible to use mixtures of two or more different water-soluble crosslinkers, for example a mixture consisting of a non-etherified and an only partially etherified melamine/formaldehyde condensation product.

Suitable crosslinkers are known commercially, for example under the name ALCOPRINT® (Ciba Specialty Chemicals).

If desired, crosslinking catalysts may additionally be used.

Suitable crosslinking catalysts for the process according to the invention are, for example, any agents customarily used as catalysts for non-ease and non-crumple finishes, as are known from Textilhilfsmittelkatalog 1991, Konradin Verlag R. Kohlhammer, Leinfelden-Echterdingen 1991. Examples of suitable crosslinking catalysts are inorganic acids, for example phosphoric acid; Lewis acids, for example zinc chloride, zirconium oxychloride, NaBF₄, AlCl₃, MgCl₂; ammonium salts, for example ammonium sulfate, ammonium chloride; or hydrohalides, especially hydrochlorides of organic amines, for example CH₃—CH₂—CH₂—NH—CH₃.HCl. Preference is given to the use of ammonium salts or magnesium-containing Lewis acids and, especially, to the use of ammonium chloride or magnesium chloride.

To increase the softness of the dyed or printed fibre material and thus to obtain a particular handle, the dyeing preparations used according to the invention may additionally comprise a fabric softener. Fabric softeners are known in the textile industry. They are non-ionic, anionic-active, cationic or amphoteric softeners. Emulsions of silicones, mostly high-molecular-weight α,ω-dimethylpolysiloxane, occupy a special position. Fabric softeners based on silicone emulsions are preferred. Such fabric softeners are commercially available, for example under the name AVIVAN® or ULTRATEX® (Ciba Specialty Chemicals).

If desired, the dyeing preparation may additionally comprise acid donors such as butyrolactone or sodium hydrogen phosphate, preservatives, sequestering agents, emulsifiers, water-insoluble solvents, oxidising agents or deaerating agents.

Suitable preservatives are especially formaldehyde-yielding agents, such as, for example, paraformaldehyde and trioxane, especially aqueous, approximately from 30 to 40% by weight formaldehyde solutions; suitable sequestering agents are, for example, nitrolotriacetic acid sodium, ethylenediaminetraacetic acid sodium, especially sodium polymetaphosphate, more especially sodium hexametaphosphate; suitable emulsifiers are especially adducts of an alkylene oxide and a fatty alcohol, especially an adduct of oleyl alcohol and ethylene oxide; suitable water-insoluble solvents are high boiling, saturated hydrocarbons, especially paraffins having a boiling range of approximately from 160 to 210° C. (so-called white spirit); a suitable oxidising agent is, for example, an aromatic nitro compound, especially an aromatic mono- or di-nitro-carboxylic or -sulfonic acid which may be in the form of an alkylene oxide adduct, especially a nitrobenzenesulfonic acid; and suitable deaerating agents are, for example, high boiling solvents, especially turpentine oils, higher alcohols, preferably C₈-C₁₀alcohols, terpene alcohols or deaerating agents based on mineral oils and/or silicone oils, especially commercial formulations composed of approximately from 15 to 25% by weight of a mineral oil and silicone oil mixture and approximately from 75 to 85% by weight of a C₈ alcohol, such as, for example, 2-ethyl-n-hexanol.

The dyeing preparations can be applied to the fibre materials by various methods, especially in the form of aqueous dye baths and printing pastes. They are especially suitable for dyeing by the pad dyeing process and for printing. Other suitable processes are the foam dyeing process, the spray dyeing process and printing by the ink-jet printing process or by the chromojet process which is used, for example, in carpet printing.

The antimicrobial SiO_{z} flakes of the present invention are used in the dyeing baths or printing pastes in general in amounts of from 0.001 to 15% by weight, especially from 0.01 to 1% by weight, based on the weight of the material being treated, and from 0.05 to 200 g, especially from 1.0 to 100 g, of the antimicrobial SiO_{z} flakes of the present invention per kg of printing paste have proved advantageous.

The printing paste usually comprises from 1 to 400 g, especially from 20 to 250 g, of binder per kg of printing paste.

In addition to comprising the antimicrobial SiO_{z} flakes and binder, the printing paste advantageously comprises thickeners of synthetic origin, such as, for example, those based on poly(meth)acrylic acids, poly(meth)acrylamides, and their copolymers and terpolymers.

Thickeners based on potassium or sodium salts of poly(meth)acrylic acids are preferably used since the addition of ammonia or ammonium salts can advantageously be partially or completely dispensed with when such thickeners are used.

Examples of other thickeners are commercial alginate thickenings, starch ethers, locust bean flour ethers and cellulose ethers. Suitable cellulose ethers are, for example, methyl-, ethyl-, carboxymethyl-, hydroxyethyl-, methylhydroxyethyl-, hydroxypropyl- and hydroxypropyl-methyl-cellulose. Suitable alginates are especially alkali metal alginates and preferably sodium alginate.

In printing of the fibre material, the printing paste is applied directly to the fibre material over the entire surface or in places, advantageously using printing machines of conventional design, for example intaglio printing machines, rotary screen printing machines, roller printing machines and flat screen printing machines.

After being printed, the fibre material is advantageously dried, preferably at temperatures of from 80 to 120° C.

Fixing of the print can then be carried out, for example, by a heat treatment, which is preferably performed at a temperature of from 120 to 190° C. Fixing preferably takes from 1 to 8 minutes in that case.

Fixing can also be carried out, however, with ionising radiation or by irradiation with UV light.

When ultraviolet radiation is used, the presence of a photoinitiator is generally required. The photoinitiator absorbs the radiation in order to produce free radicals that initiate the polymerisation. Suitable photoinitiators are known to the person skilled in the art.

The process according to the invention is suitable for dyeing or printing very diverse fibre materials, such as wool, silk, cellulose, polyacrylonitrile, polyamide, aramide, polyolefins, for example polyethylene or polypropylene, polyesters or polyurethane.

Preference is given to fibre materials containing cellulose. Suitable fibre materials containing cellulose are materials that consist entirely or partially of cellulose. Examples are natural fibre materials, such as cotton, linen or hemp, regenerated fibre materials, such as, for example, viscose, polynosic or cuprammonium rayon. Also suitable are mixed fibre materials containing cellulose, that is to say, mixtures of cellulose and other fibres, especially cotton/polyester fibre materials.

Wovens, knits or webs of those fibres are mainly used.

The process of this invention makes it possible to obtain with antimicrobial SiO_{z} flakes of the present invention finished textile materials having long lasting efficacy.

It is also possible to incorporate the antimicrobial SiO_{z} flakes of the present invention in nonwovens.

“Non-woven” is a type of fabric that is not spun and woven into a cloth, but instead bonded together. According to the ISO definition it is a manufactured sheet, web, or batt of directionally or randomly orientated fibers, bonded by friction, and/or adhesion.

Nonwoven textiles are widely used in disposable as well as durable goods, such as baby diaper, feminine hygiene, adult incontinence, wipers, bed linings, automotive industries, medical face masks, air and water filtration, home furnishing and geotextiles. Such materials can be fabricated by different techniques, such as spunbonding, melt blown, carded thermal bonding and carded chemical bonding, dry and/or wet laid and needlefelts. Because of the nature of such applications the market is increasingly demanding products with specific properties such as antimicrobial efficacy.

Amongst various nonwoven products, materials made by spunbonding and melt blown techniques have some unique properties and are becoming more and more important because of advantages in manufacturing as well as in product properties. Spunbond nonwovens can be made directly from thermoplastic polymers such as polypropylene, polyethylene, polyester and nylon. This process offers lower manufacturing cost, improved processability and performance in the final product such as coverstock for disposable baby diapers, feminine hygiene and adult incontinence. Spunbond nonwovens can also be used as durable products such as geotextiles and roof membranes. Characterised by a large surface area and small pore size, melt blown nonwovens differ from traditional spunbonds in their lower fiber denier and fineness. But similarly, melt blown nonwovens are also manufactured by directly extruding thermoplastic polymers, especially high melt flow polypropylene. Their applications include filtration, feminine hygiene, wipers, face masks and absorbents.

The nonwovens used are preferably prepared by spun bond and melt blown processes or by carded chemical bonding, carded thermal bonding, dry and/or wet laid and needlefelts. Accordingly, the antimicrobial SiO_{z} flakes of the present invention can also be used for the production of antimicrobial textile articles.

In said aspect the invention provides a fibrous textile article comprising antimicrobial SiO_{z} flakes of the present invention, said antimicrobial SiO_{z} flakes being present in an amount sufficient to impart antimicrobial properties to said article. The content of the antimicrobial SiO_{z} flakes suitably ranges from 0.001 to 10 wt %, preferably 0.01 to 1 wt %.

Textile articles comprising the antimicrobial SiO_{z} flakes of the present invention, particularly woven and non-woven hydrophilic fabrics, exhibit outstanding antimicrobial resistance with respect to pathogens such as bacteria, viruses, yeast and algae, are resistant to degradation upon exposure to sunlight (ultraviolet light) and maintain their excellent antimicrobial properties even after a number of launderings.

The present invention is also directed to an optically dear lens having antimicrobial properties comprising the antimicrobial SiO_{z} flakes according to the present invention, especially antimicrobial SiO_{z} flakes comprising silver.

As used herein, the phrase “optically clear” refers to a lens that has optical clarity comparable to currently available commercial lenses, e.g. etafilcon A, balafilcon A, and the like.

The optical clearness of the antimicrobial SiO_{z} flakes according to the present invention can be controlled by the following parameters:

Pore size of the porous SiO_{z} flakes, which is especially in the range of I to 20 nm, very especially 2 to 10 nm, i.e. the particle size of the silver nanoparticles, which is especially in the range of 1 to 20 nm, very especially 2 to 10 nm.

Temperature during contact of the AgNO₃ solution and the SiO_{z} flakes as well as

the calcination temperature of the antimicrobial SiO_{z} flakes, which is generally below 900° C., especially below 600° C., very especially 200 to 600° C.

The term “lens” refers to opthalmic devices that reside in or on the eye. These devices can provide optical correction or may be cosmetic. The term lens includes but is not limited to soft contact lenses, hard contact lenses, intraocular lenses, overlay lenses, ocular inserts, and optical inserts. Typical hard contact lenses are made from polymers which include but are not limited to polymers of poly (methyl) methacrylate, silicone acrylates, fluoroacrylates, fluoroethers, polyacetylenes, and polyimides, where the preparation of representative examples may be found in JP 200010055, JP 6123860, and U.S. Pat. No. 4,330,383. Typical soft contact lenses are made from silicone elastomers, or hydrogels, such as but not limited to silicone hydrogels and fluorohydrogels. The preparation of representative soft contact lenses may be found in U.S. Pat. No. 5,710,302, WO94/21698, EP-A406161, JP2000016905, U.S. Pat. No. 5,998,498, and U.S. Pat. No. 6,087,415. Examples of commercially available soft contact lenses include but are not limited to etafilcon A, genfilcon A, lenefilcon A, polymacon, andlotrafilcon A. Intraocular lenses of the invention can be formed using known materials. For example, the lenses may be made from a rigid material including, without limitation, polymethyl methacrylate, polystyrene, polycarbonate, or the like, and combinations thereof. Additionally, flexible materials may be used including, without limitation, hydrogels, silicone materials, acrylic materials, fluorocarbon materials and the like, or combinations thereof. Typical intraocular lenses are described in WO0026698, WO0022460, WO9929750, WO9927978, and WO0022459. All of the aforementioned lenses may be coated with a number of agents that are used to coat lens. For example, the procedures, compositions, and methods of U.S. Pat. No. 6,087,415 may be used and this patent is hereby incorporated by reference for those procedures, compositions, and methods. The antimicrobial SiO_{z} flakes comprising silver can be added to the monomer mix of the other components. The resulting mixture is charged to molds and cured,

The amount of silver in the lens is greater than 0.01 weight percent, where the percentage is based the weight of the components of the un-hydrated monomer. The weight percentage of silver is about 0.01 to about 0.3 weight percent, more preferably, about 0.02 to about 0.2 weight percent, and most preferably about 0.03 to about 0.1 weight percent.

The phrase “antimicrobial properties” refers to lenses that exhibit one or more of the following properties, the inhibition of the adhesion of bacteria or other microbes to the lenses, the inhibition of the growth of bacteria or other microbes on lenses, and the killing of bacteria or other microbes on the surface of lenses or in a radius extending from the lenses. Particularly, preferably, the lenses of the invention exhibit at least a 1-log reduction (>90% inhibition) of viable bacteria or other microbes, most particularly preferably, about a 2-log reduction (>99% inhibition) of viable bacteria or other microbes in in vitro tests. Such bacteria or other microbes include but are not limited to those organisms found in the eye, particularly Pseudomonas aeruginosa, Acanthanmoeba, Staph. aureus, E. coli, Staphyloccus epidermidus, and Serratia marcesens.

Yet still further, the invention includes a lens case having antimicrobial properties, comprising the antimicrobial SiO_{z} flakes according to the present invention, especially the antimicrobial SiO_{z} flakes comprising silver. The term lens case refers to a container that is adapted to define a space in which to hold a lens when that lens is not in use. This term includes packaging for lenses, where packaging includes any unit in which a lens is stored after curing. Examples of this packaging include but are not limited to single use blister packs and the like. One such container is illustrated in FIG. 3 of U.S. Pat. No. 5,515,117. The antimicrobial SiO_{z} flakes can be incorporated in the lens container 22, the cover 24, or the lens basket 26, where they are preferably incorporated into the lens container or the lens basket. (numbers refer to U.S. Pat. No. 5,515,117).

Aside from the antimicrobial SiO_{z} flakes comprising silver, the container components may be made of a transparent, thermo-plastic polymeric material, such as polymethylmethacrylate, polyolefins, such as poly-ethylene, polypropylene and the like; polyesters, polyurethanes; acrylic polymers, such as polyacrylates and polymethacrylates; polycarbonates and the like and is made, e.g., molded, using conventional techniques as a single unit. In the same manner as the lenses of the invention, the antimicrobial SiO_{z} flakes comprising silver, can be added to the monomer mix of the other components. The resulting mixture is charged to molds and cured. Preferably, activated silver is present in any or all of the lens case components at about 0.01 to about 10 weight percent (based on the initial monomer mix), more preferably about 0.05 to about 3.0 percent.

The present invention is also directed to dental appliances comprising a polymeric material incorporating the antimicrobial SiO_{z} flakes. The antimicrobial SiO_{z} flakes constitute between about 0.5 to 50.0 percent of the total weight of the polymeric material. The polymeric material is preferably a coating comprising the antimicrobial SiO_{z} flakes. The dental appliance is preferably a dental bracket, or an arch wire.

The Examples that follow illustrate the invention without limiting the scope thereof. Unless otherwise indicated, percentages and parts are percentages and parts by weight, respectively.

### EXAMPLES

### Example 1

A solution of 17.0 g (0.1 mol) AgNO₃ in ice-cold de-ionized water (200 ml) is prepared with stirring under an atmosphere of nitrogen. 2.0 g of porous SiO_{z} flakes (z≈1.4-1.6; BET=716 m²/g) obtained in analogy to example 1 of WO2004/065295 are added slowly with continuous stirring. The suspension is stirred for 7 days at room temperature, filtered, rinsed nitrate free with de-ionized water and methanol. The residue is dried in vacuo at 35° C. and calcined at 600° C.

The silver containing silicon oxide flakes are characterized by X-ray diffraction. Elemental analysis shows a silver content of 0.46%. The surface area derived from BET measurements is 704 m²/g.

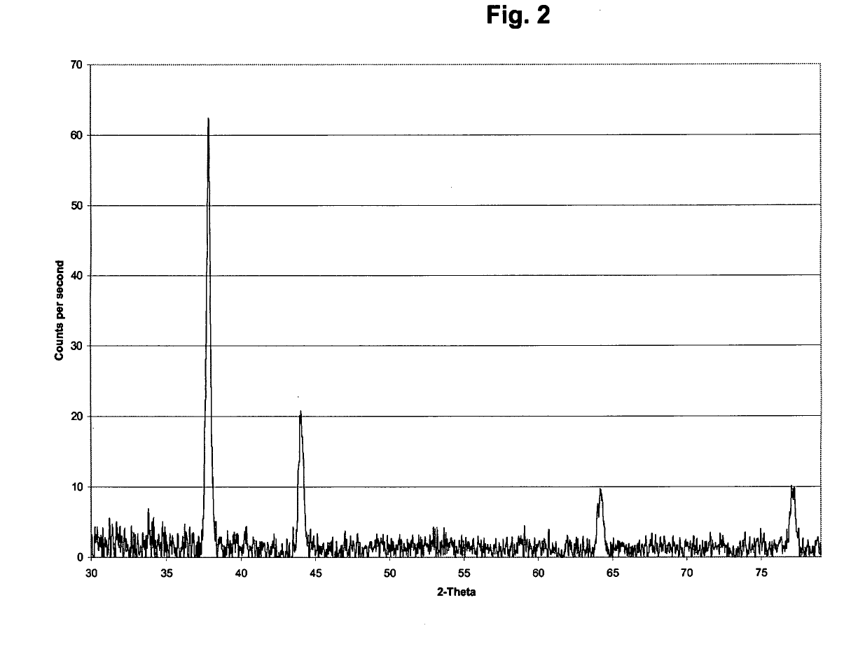

FIG. 2 is an X-ray diffraction pattern of the silver coated flakes SiO_{z} flakes of Example 1. No peaks of AgNO₃ or Ag₂O are present.

The microbicidal activity of the obtained silver containing silicon oxide flakes is determined according to CG 161/European Standard method EN 1040. The silver containing silicon oxide flakes exhibit excellent microbiocidal activity against S. aureus and E. coli. (>5 log after 5 minutes) at a suspension concentration of 1%. Silver-containing zeolites with a silver content of 2.1-2.2% don't show any activity after 5 minutes, when tested in a suspension concentration of up to 2.5%.

### Example 2

1.0 g of porous SiO_{z} are suspended in 75 ml of methanol and cooled to −50° C. A solution of 0.25 g (1.5 mmol) AgNO₃ in 75 ml methanol is prepared, completely homogenized in an ultrasonic bath and added dropwise to the SiO_{z} suspension at −50° C. under continued stirring.

The suspension is stirred for 4 hours at −50° C., filtered and rinsed nitrate free with methanol. The residue is dried in vacuo at 60° C. and optionally calcined at 400 to 600° C.

The silver on the SiO_{z} flakes is characterized by X-ray diffraction. Elemental analysis proves a silver content of 0.5%. The surface area derived from BET measurements is obtained to be 1000 m²/g. The silver coated flakes obtained in the process described in Example 2 show excellent microbicidal activity against S. aureus and E. coli. (>5 log after 5 minutes) at a suspension concentration of 1%.

### Examples 3 to 5

In analogy to example 2 the following silver coated flakes SiO_{z} flakes (z=1.4-1.6) are obtained:

| | Ag Content | BET Surface Area |
|---|---|---|
| Example | [% by weight] | [m²/g] |
| 3 | 0.5 | 1002 |
| 4 | 0.6 | 750 |
| 5 | 0.8 | 800 |

### Example 6

0.35 g of porous SiO_{z} are suspended in 100 ml of methanol and cooled to −50° C. A solution of 0.25 g (1.5 mmol) AgNO₃ in 20 ml de-ionized water is prepared, mixed with 0.35 ml of a 25% NH₃ solution and subsequently replenished to 50 ml with methanol. This solution is added dropwise to the SiO_{z} suspension mentioned above at −50° C. under continued stirring. The suspension is stirred for 2 hours at −50° C., filtered and rinsed nitrate free with cooled methanol. The residue is dried in vacuo at 60° C.

Elemental analysis shows a silver content of 6.0% wt Ag.

### Example 7

1 g of non-porous SiO_{z} flakes (obtained in analogy to the process described in Example 1 of WO03/068868; (z≈1.4-1.6) are suspended in 60 ml of methanol and cooled to −60° C. A solution of 60 ml 0.5 M aqueous AgNO₃ is prepared and added dropwise to the SiO_{z} suspension with continued stirring at −60° C. for 2 hours. The suspension is filtered, washed thoroughly with cooled de-ionised water and with cooled methanol to get a nitrate free filtrate. The residue is dried in vacuo at 60° C. The silver coated flakes are optionally heated in air at 400° C. for 20 hours.

Elemental analysis shows a silver content of 0.44% wt Ag. The silver coated flakes obtained in the process described in Example 7 show excellent microbicidal activity against S. aureus and E. coli (>5 log after 5 minutes) at a suspension concentration of 1%.

### Example 8

6.7 g of porous SiO_{z} flakes (obtained in analogy to the process described in Example 1 of WO03/068868; (z≈1.4-1.6) are suspended in 500 ml of de-ionized water at room temperature. A solution of 380 mg AgNO₃ in 50 ml de-ionized water is prepared and added dropwise to the SiO_{z} suspension with continued stirring for 3 hours. The black suspension is filtered, washed thoroughly with de-ionised water and with methanol. The residue is dried in vacuo at 60° C. The silver coated flakes are optionally heated in air at 600° C. for 3 hours to yield a colourless product. Elemental analysis exhibits a silver content of 0.82% wt Ag.

### Example 9

1.5 g of porous SiO_{z} flakes (obtained in analogy to the process described in Example 1 of WO03/068868; z≈1.4-1.6) are suspended in 25 ml of de-ionized water at room temperature. A solution of 0.32 g CuCl₂ in 15 ml de-ionized water is prepared and added dropwise to the SiO_{z} suspension with continued stirring. The suspension in heated to 80° C. for 16 hours. To the cooled mixture a solution of 0.47 g hydrazine hydrate in 15 ml of de-ionized water is added dropwise. The suspension in heated to 80° C. for 16 hours. The dark mixture is filtered and washed thoroughly with de-ionised water and with methanol. The residue is dried in vacuo at 60° C. The product is optionally heated in air at 500° C. for 3 hours to yield 1.40 g.

Elemental analysis exhibits a copper content of 9.62% wt Cu.

### Example 10

The same procedure as for Example 11 with 0.36 g NaBH₄ instead of hydrazine hydrate yields a grey product with 7.47% wt Cu.

### Example 11

4.8 g of porous SiO_{z} flakes (obtained in analogy to the process described in Example 1 of WO03/068868; z≈1.4-1.6) are suspended in 150 ml of de-ionized water at room temperature. A solution of 0.89 PdCl₂ and 1.6 g NaCl in 50 ml de-ionized water is prepared and added dropwise to the SiO_{z} suspension with continued stirring. The suspension in heated to 75° C. for 1 hour. To the cooled mixture a solution of 0.25 9 hydrazine hydrate in 25 ml of de-ionized water is added dropwise. The black suspension is filtered and washed thoroughly with de-ionised water and with methanol. The residue is dried in vacuo at 60° C. The palladium coated flakes are optionally heated in air at 600° C. for 2 hours to yield 4.86 g of a brown product. Elemental analysis exhibits a palladium content of 9.60% wt Pd.

### Example 12

0.08 g nickel acetate tetrahydrate are solved in 30 ml of de-ionized water. 0.51 9 of porous SiO_{z} flakes are added with stirring. A solution of 10 mg NaBH₄ in 30 ml de-ionized water is prepared and added dropwise to the SiO_{z} suspension with continued stirring. The suspension in heated to 80° C. for 20 hours. The cooled mixture is then filtered and washed thoroughly with de-ionised water and with methanol. The residue is dried in vacuo at 60° C. Optional heat treatment in air at 600° C. for 2 hours yields 0.47 9 of a light green product.

1. A SiO_{z} flake, wherein 0.70≦z≦2.0 comprising an organic or inorganic antimicrobial compound or composition.

2. The SiO_{z} flake according to claim 1, wherein the organic antimicrobial compound or composition is selected from dimethyldimethylol hydantoin, methylchloroisothiazolinone/methylisothiazolinone, imidazolidinyl urea, diazolidinyl urea, benzyl alcohol, 2-bromo-2-nitropropane-1,3-diol-formalin (formaldehyde), iodopropenyl butylcarbamate, chloroacetamide, methanamine, methyldibromonitrile glutaronitrile, glutaraldehyde, 5-bromo-5-nitro-1,3-dioxane, phenethyl alcohol, o-phenylphenol/sodium o-phenylphenol, sodium hydroxymethylglycinate, polymethoxy bicyclic oxazolidine, dimethoxane, thimersal, dichlorobenzyl alcohol, captan, chlorphenenesin, dichlorophene, chlorbutanol, glyceryl laurate,halogenated diphenyl ethers,2,4,4′-trichloro-2′-hydroxy-diphenyl ether, 4,4′-dichloro-2′-hydroxydiphenyl ether, 2,2′- dihydroxy-5,5′-dibromo-diphenyl ether, phenol, 2-methyl phenol, 3-methyl phenol, 4-methyl phenol, 4-ethyl phenol, 2,4-dimethyl phenol, 2,5-dimethyl phenol, 3,4-dimethyl phenol,2,6-dimethyl phenol, 4-n-propyl phenol, 4-n-butyl phenol, 4-n-amyl phenol, 4-tert-amyl phenol, 4-n-hexyl phenol, 4-n-heptyl phenol, mono- and poly-alkyl and aromatic halophenols, p-chlorophenol, methyl p-chlorophenol,ethyl p-chlorophenol, n-propyl p-chlorophenol, n-butyl p-chlorophenol, n-amyl p-chlorophenol, sec-amyl p-chlorophenol, cyclohexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, o-benzyl p-chlorophenol, o-benxyl-m-methyl p-chlorophenol, o-benzyl-m, m-dimethyl p-chlorophenol, o-phenylethyl p-chlorophenol, o-phenylethyl-m-methyl p-chlorophenol, 3-methyl p-chlorophenol, 3,5-dimethyl p-chlorophenol, 6-ethyl-3-methyl p-chlorophenol, 6-n-propyl-3-methyl p-chlorophenol, 6-iso-propyl-3-methyl p-chlorophenol, 2-ethyl-3,5-dimethyl p-chlorophenol, 6-sec-butyl-3-methyl p-chlorophenol, 2-iso-propyl-3,5-dimethyl p-chlorophenol, 6-diethylmethyl-3-methyl p-chlorophenol, 6-iso-propyl-2-ethyl-3-methyl p-chlorophenol, 2-sec-amyl-3,5-dimethyl p-chlorophenol, 2-diethylmethyl-3,5-dimethyl p-chlorophenol, 6-sec-octyl-3-methyl p-chlorophenol, p-chloro-m-cresol, p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2-phenyl phenol.4-chloro-2-methyl phenol,4-chloro-3-methyl phenol, 4-chloro-3,5-dimethyl phenol, 2,4-dichloro-3,5-dimethylphenol, 3,4,5,6-terabromo-2-methylphenol, 5-methyl-2-pentylphenol, 4-isopropyl-3-methylphenol,para-chloro-meta-xylenol (pcmx), chlorothymol, phenoxyethanol, phenoxyisopropanol, 5-chloro-2-hydroxydiphenylmethane, resorcinol, methyl resorcinol, ethyl resorcinol, n-propyl resorcinol, n-butyl resorcinol, n-amyl resorcinol, n-hexyl resorcinol, n-heptyl resorcinol, n-octyl resorcinol, n-nonyl resorcinol, phenyl resorcinol, benzyl resorcinol, phenylethyl resorcinol, phenylpropyl resorcinol, p-chlorobenzyl resorcinol, 5-chloro 2,4-dihydroxydiphenyl methane, 4′-chloro 2,4-dihydroxydiphenyl methane, 5-bromo 2,4-dihydroxydiphenyl methane, 4′-bromo 2,4-dihydroxydiphenyl methane, 2,2′-methylene bis(4-chlorophenol), 2,2′-methylene bis(3,4,6-trichlorophenol), 2,2′-methylene bis(4-chloro-6-bromophenol), bis(2-hydroxy-3,5-dichlorophenyl)sulphide, bis(2-hydroxy-5-chlorobenzyl)sulphide, methylparaben, propylparaben, butylparaben, ethylparaben, isopropylparaben, isobutylparaben, benzylparaben, sodium methylparaben, sodium propylparaben, halogenated carbanilides, 3,4,4′-trichlorocarbanilides, 3-trifluoromethyl-4,4′-dichlorocarbanilide, 3,3′,4-trichlorocarbanilide, chlorohexidine and its digluconate, diacetate and dihydrochloride, undecenoic acid, hexetidine, poly(hexamethylenebiguanide) hydrochloride, thiabendazole, 10,10′ oxybisphenoxyarsine, tebuconazole, tolnaftate, zinc bis-(2-pyridinethiol-1-oxide), 2n-octyl-4-isothiazolin-3-one, 4,5-dichloro-octyl-4-isothiazoline, N-butyl-benzisothiazoline, 3-iodo-2-propinylbutylcarbamate, methyl-1H-benzimidazol-2-ylcarbamate and mixtures thereof.

3. The SiO_{z} flake according to claim 1, wherein the inorganic antimicrobial compound or composition comprises an antimicrobial metal salt which comprises salts of metals selected from the group consisting of Mn, Ag, Au, Zn, Sn, Fe, Cu, Al, Ni, Co, Ti, Zr, Cr, La, Bi, K, Cd, Yb, Dy, Nd, Ce, Tl, Pr, and combinations thereof.

4. The SiO_{z} flake according to claim 1, wherein the metal salts are selected from the group consisting of fluorides, aspartates, gluconates, iodides, oxides, nitrites, nitrates, phosphates, pyrophosphates, sulfides, mercaptopyridine- oxides-, nicotinates, and nicotinamides, hinokitiol, acetates, ascorbates, chlorides, benzoates, citrates, fumarates, gluconates, glutarates, lactates, malates, malonates, salicylates, succinates, sulfates, undecylates, and combinations thereof.

5. The SiO_{z} flake according to claim 1, wherein the inorganic antimicrobial compound or composition comprises a metal selected from Mn, Ag, Au, Zn, Sn, Fe, Cu, Al, Ni, Co, Ti, Zr, Cr, La, Bi, K, Cd, Yb, Dy, Nd, Ce, Tl, Pr and combinations therof.

6. The SiO_{z} flake according to claim 4, wherein the SiO_{z} flakes contain the antimicrobial metal salt in an amount of 0.001 to 20.0 percent by weight.

7. An antimicrobial composition, comprising a high weight organic material and the antimicrobial SiO_{z} flakes according to claim 1.

8. An antimicrobial composition according to claim 7, wherein the high molecular weight organic material is a thermoplastic or thermosetting resin.

9. (canceled)

10. An antimicrobial product, comprising the antimicrobial SiO_{z} flakes according to claim 1, wherein the product is a personal care product, household care product, an industrial or hospital product, a textile article, a fiber material, a paper material, a paper coating, an adhesive, a decorative coating, an industrial coating, a powder coating, or a paint.

11. (canceled)

12. A process for preparing the antimicrobial SiO_{z} flakes according to claim 5, comprising
a) suspending the SiO_{z} flakes in a solvent, wherein z′<2,
b) adding a solvent soluble antimicrobial metal salt and optionally a reducing agent to the solution, and
c) isolation of the metal containing SiO_{z} flakes, wherein 0.70≦z≦2.0.

13. The SiO_{z} flake according to claim 1, which is a porous SiO_{z} flake.

14. The SiO_{z} flake according to claim 1, wherein 0.95≦z≦2.0.

15. The SiO_{z} flake according to claim 5, wherein metal is selected from silver, gold, copper, zinc and combinations therof.

16. The SiO_{z} flake according to claim 5, which is a porous SiO_{z} flake.

17. The SiO_{z} flake according to claim 5, wherein 0.95≦z≦2.0.

18. An antimicrobial composition according to claim 8, wherein the thermoplastic or thermosetting resin is selected from the group consisting of polyethylene, polypropylene, polyvinyl chloride, acrylonitrile-butadiene-styrene copolymer, nylons, polyesters, unsaturated polyesters, polyvinylidene chloride, polyamides, styrene-acrylonitrile copolymers, polystyrene, polymethyl methacrylate, polyacryl nitrile, polyethylene terephthalate, polyacetals, polyvinyl alcohol, polycarbonate, acrylic resins, fluoroplastics, polyurethane, thermoplastic polyurethane, phenolic resins, meramine resins, epoxy resins, rayon, urea formaldehyde resin, cuprammonium rayon, acetates, triacetates, vinylidene, natural rubber, synthetic rubber, alkyl resin and aminoalkyd resin.

19. An antimicrobial composition according to claim 7, which comprises a paint, lacquer or varnish.

20. An antimicrobial product according to claim 10, wherein the antimicrobial SiO_{z} flakes comprise porous SiO_{z} flakes
